(19) United States
(12) Patent Application Publication
(10) Pub. No.: US 2020/0003762 A1
(43) Pub. Date: Jan. 2, 2020

(54) Diagnostics Platform for Mitochondrial Dysfunctions/Diseases

(71) Applicant: BioElectron Technology Corporation, Mountain View, CA (US)

(72) Inventor: Stephen J. Brown, Woodside, CA (US)

(21) Appl. No.: 16/024,486

(22) Filed: Jun. 29, 2018

(51) Int. Cl. G01N 33/50; G06F 19/24; G06F 19/28

(52) U.S. Cl. CPC G01N 33/5079; G06F 19/24; G06F 19/28; G01N 2800/60; G01N 2800/52; G01N 2800/2821; G01N 2800/323; G01N 2800/104; G01N 2800/042; G01N 2800/302; G01N 2800/304; G01N 2800/305; G01N 2800/285

(57) ABSTRACT

The present invention concerns machine learning based methods and systems for diagnosing and treating genetic diseases characterized by mitochondrial dysfunctions. A library of reference learning models is developed based on in vitro reference samples obtained from cell-cultures exposed to specific mitochondrial inhibitors. Each model is able to predict a specific labeled mitochondrial dysfunction induced in the cell-culture by the inhibitor/stressor. The reference models are then applied to target samples drawn in vivo from target subjects who are known to have specific genetic mitochondrial diseases. A mapping is developed between mitochondrial dysfunctions predicted in the subjects and their known mitochondrial diseases. This mapping and the reference models are then applied to a clinical sample of an undiagnosed patient in whom a diagnosis of a mitochondrial dysfunction and an associated mitochondrial disease is made. If there is a known rescuer for the mitochondrial dysfunction, it may be recommended in a personalized, targeted therapy.

### CROSS-REFERENCE TO RELATED APPLICATIONS

The present application is related to U.S. patent application Ser. No. 15/785,415 filed on Oct. 16, 2017 under the title “Redox-related context adjustments to a bioprocess monitored by learning systems and methods based on redox indicators”, which is a continuation-in-part of U.S. patent application Ser. No. 15/675,364 filed on Aug. 11, 2017 under the title “Distributed systems and methods for learning about a bioprocess from redox indicators and local conditions”.

The present application is also related to U.S. Provisional Patent Application 62/544,749 filed on Aug. 11, 2017 under the title “Monitoring and control of electron balance in bioreactor systems” and U.S. Provisional Patent Application 62/621,394 filed on Jan. 24, 2018 under the title “Improved diagnostic systems and methods for poorly characterized syndromes and biological entities based on bioprocess learning models”.

All the above numbered applications are incorporated by reference herein in their entireties.

### FIELD OF THE INVENTION

The present invention relates to apparatus and methods for applying machine learning algorithms to bioprocesses for diagnosing mitochondrial dysfunctions based on biomarker measurements initially obtained from biological samples in vitro and then targeted on subjects with genetic mitochondrial diseases in vivo.

### BACKGROUND OF THE INVENTION

By most definitions, all entities or systems undergoing a biological process or a bioprocess are considered to be alive.

Living biological entities range from biological systems, e.g., biomasses in controlled bioreactors, to living organisms. The latter include animals and plants. Often, biological entities at this level are viewed in the context of their environments or local conditions that are either conducive to their existence or not.

Living entities on planet Earth can be broken down into bacteria, archaea and eukaryotes. Their sizes, from smallest to largest, span many orders of magnitude. The bioprocesses that these biological entities undergo are extremely varied and highly complex. The study of biological entities at this level belongs to the fields of biology, ecology, zoology and botany. Despite the truly remarkable amount of differentiation among biological entities, they do share common structures and operating principles. One such operating principle is that all biological entities depend on harvesting external energy sources to stay alive. In terms of common structures, all biological entities, except perhaps viruses, are made up of a smallest basic living component: the cell. While being the smallest units of life, cells also coincide with the smallest living biological entities of interest: bacteria.

At the cell level, life is again found to exhibit myriads of complex structures and processes. The processes of interest happen here on much shorter time scales than at the higher level of multi-cellular biological entities. A new set of common operating principles and shared structures are found at the cell level.

In particular, processes occurring at the cell level are described by molecular biology and biochemistry. They can be understood in terms of biochemical structures and reactions. The most important biochemical reactions include construction, replication, feeding, repair, energy regulation, and carrying out of primary cell functions (dependent on cell type).

Below the cell level is the realm of processes and structures operating on still shorter time scales. It is the level of physical organic chemistry and, ultimately, quantum chemistry and quantum physics. The latter govern the actions of atoms and of small molecules by rules that transcend classical logic and assumptions. Still, common structures and processes are found even at this level.

Many approaches and techniques for understanding the structures and processes of physical organic chemistry have been proposed over the past fifty years. One prominent modeling approach attempting to explain the relationship between specific structures and activities is the Quantitative Structure-Activity Relationship (QSAR) model. QSAR was introduced by Corwin Hansch et al. in 1962. An excellent text describing this contribution and the consequent approaches developed from it is provided by Hugo Kubinyi, “QSAR: Hansch Analysis and Related Approaches”, Methods and Principles in Medicinal Chemistry, New York, 1993.

More recent 3D QSAR and Comparative Molecular Field Analysis (CoMFA) models have attempted to apply quantum-chemical tools to determine chemical reactivity at the level of physical organic chemistry. These models track the formation of hydrogen bonds, proton movement/hopping, electron exchanges and/or oxidation-reduction (redox) reactions as well as steric effects. The latter affect ligand binding preferences and are also related 3D alignment effects. Although the practice of 3D QSAR is inherently limited to local models at this level of study, it can be expected to make further progress. Specifically, the expansion of published databases such as ChEMBL and PubChem along with annotations and 3D alignment protocols, may continue to provide better validated physical organic chemistry models for both screening (e.g., drug or toxic substance screening) and machine learning applications in this field. An excellent summary of the present state of the art in this realm is afforded by Cherkasov, et al., “QSAR Modeling: Where have you been? Where are you going to?”, J. Med. Chem., Volume 57, No. 12, Jun. 26, 2014, pp. 4977-5010 and the numerous references cited therein.

Systems biology examines life as it builds on top of the low level of physical organic chemistry, which is in the purview of 3D QSAR and other Field Models addressed above. Systems biology is further informed by data collected in the various -omes, and in particular the genome and the proteome. In examining the Genome-Protein-Reaction (GPR) chain, systems biology brings to bear traditional tools of applied mathematics and linear algebra. It has attempted to deploy these tools to model biology in terms of metabolic networks, elements, reactions, fluxes as they act under certain constraints to achieve local equilibria or homeostasis.

The differential equations of systems biology address processes that attempt to reach the level of entire cells and even entire multi-cellular biological entities.

Systems biology has advanced the understanding of structure and biological function of simple single celled biological entities. For example, a curated genome-scale metabolic network reconstruction of Escherichia coli has been achieved in the recent past. A general review of the state of the art in systems biology is found in the textbook by Bernhard O. Palsson, “Systems Biology: Constraint-based Reconstruction and Analysis”, Dept. of Bioengineering, University of California San Diego, Cambridge University Press, 2015, and in the sources recited therein.

As is likely already clear from the above, division of life into various levels of study can only take us so far. Reconstruction from the genome information of the overall cell proteins and structure is not sufficient to tell us what regulatory processes are active at shorter time scales, e.g., in the physical chemistry layer. Thus, understanding the translation of the genetic code into proteins provides only a background against which the processes of physical chemistry unfold. Specifically, regulatory mechanisms involving the available enzymes that catalyze the millions of cell reactions occurring during each second have to be included in order to understand cell regulation. Still differently put, many of the crucial effects and regulatory mechanisms are found in the interstices between levels at which the life of the biological entity and its cells is being investigated. We also observe direct inter-level effects. Activity at the physical chemistry level, i.e., below the cell level, directly affects activity and structure at the cell level and at the level of the biological entity and its local conditions or environment.

These considerations bring back into focus the physical chemistry processes that involve the transfer of electrons and proton hopping. These processes are due to underlying field effects and molecular conformations (topology). They are generally known as reduction-oxidation reactions. Their effects occur at the cell level. Indeed, within any cell there are a number of specialized enzymes and affiliated compounds that are also involved in the regulation of these reactions. They include enzymes generally categorized as oxidoreductases, as well as their co-factors and other electron carrying molecules and/or complexes. These enzymes, co-factors and complexes participate in redox reactions to provide a critical level of balance and regulation for bioprocesses. For an introductory level review of these issues the reader is referred to standard texts, such as Bruce Alberts et al., “Molecular Biology of the Cell”, Garland Science, 5^{th} Edition, New York, 2008.

In their seminal article, Bucher, T. and Klingenberg M., “Pathways of hydrogen in the living organization”, Angewandte Chemie (Applied Chemistry), 70, pp. 225-570, 1958 examined the pathways of hydrogen in a living organization of a biological system or biological entity (bio-entity). This study addressed the interactions within the network of redox reactions extending over essential functions of living cells. The crucial nature of redox systems and redox reactions in bioprocesses occurring in biological systems and entities was thus firmly established. A redox code for classifying redox reactions was developed. The redox code consists of four principles by which biological systems and entities are organized.

The first redox principle is the use of the reversible electron accepting and donating properties in NAD and NADP to provide organization of metabolism (at or near equilibrium). The second redox principle is the use of redox electron transfers to adjust protein structure through kinetically controlled redox switches (a.k.a. S-switches or Sulphur switches) in the proteome to control tertiary structure, macromolecular interactions and trafficking, activity and function. The third redox principle is redox sensing as used in activation/deactivation cycles of redox metabolism, especially involving H₂O₂, support of spatiotemporal sequencing in differentiation and life cycles of cells and biological entities, e.g., organisms. The fourth principle is that redox networks form an adaptive system to respond to local conditions including the external environment. This adaptive system extends from micro-compartments through subcellular systems to the level of the cell and still further to tissue organization. A detailed explanation of these four redox principles is found in Jones, Dean P. et al., “The Redox Code”, Review Article appearing in Antioxidants and Redox Signaling, Vol. 0, No. 0, 2015, pp. 1-14. Further background provided by the same main author on select redox couples can be found in Jones, Dean P. et al., “Cysteine/cysteine couple is a newly recognized node in the circuitry for biologic redox signaling and control”, The FASEB Journal, Vol. 18, August, 2004, pp. 1246-1248.

Certain redox reactions and the electron balances they establish have been proposed to monitor cell status (e.g., oxidative stress) in some contexts. For example, U.S. Pat. No. 9,273,343 to Cali et al. suggests the use of compounds and methods for assaying the redox state of metabolically active cells and for measuring NAD(P)NAD(P)H balance. Tracking of certain redox reactions in conjunction with genome-scale metabolic network reconstruction has also been considered in U.S. Pat. No. 8,311,790 to Senger et al. This teaching addresses the identification of incomplete metabolic pathways to allow for the completion of genome-scale metabolic network for C. acetobutylicum. The program could thus provide a potential model of a genome-scale stoichiometric matrix that could attempt to model cell growth in silico.

The use of redox reactions for detecting certain analytes has also been investigated beyond the normal cell environment, e.g., in vitro. For example, U.S. Pat. No. 7,807,402 to Horn et al. proposes a method and reagent for detecting the presence and/or the amount of a certain analyte by a redox reaction and a fluorimetric determination. The redox reaction would be monitored here by a certain redox indicator. The oxidizing or reducing system would act directly on the redox indicator or via a mediator. The presence of the analyte would result in a reduction or oxidation of the redox indicator, which would allow for a qualitative or quantitative determination. U.S. Pat. No. 9,605,295 to Yau suggests an ultrasensitive and selective system and method for detecting certain reactants of the chemical/biochemical reaction catalyzed by an oxidoreductase. The action of the electrical field is suggested to facilitate the interfacial electron transfer between oxidoreductase and the working electrode of his electrochemical system by the quantum mechanical tunneling effect. Additional teachings of Yau involving bio-reactive systems and their voltage-controlled metabolism are found in U.S. Pat. Appl. No. 2016/0333301.

U.S. Pat. Appl. No. 2016/0166830 to Avent et al. illustrates the difficulties in devising systems, devices and methods to selectively provide antioxidant or pro-oxidant effects to control free radical damage in an organism. The therapeutic electron and ion transfer via half-cell involves providing electrodes, which may include syringe needles, to establish conductive paths to or from the organism, e.g., a human patient.

In principle, a needle-type testing apparatus could be miniaturized and improved by leveraging MEMS technologies for specific analytes. Examples of such apparatus and methods proposed to measure certain chemical species in biological samples, including certain specific reduction-oxidation potentials are found in the literature. The reader is referred to Hyoung-Lee, W. et al., “Needle-type environmental microsensors: design, construction and uses of microelectrodes and multi-analyte MEMS sensor arrays”, Measurement Science and Technology, Vol. 22, March 2011 (22 pgs.) and to Lee, Jin-Hwan et al., “MEMS Needle-type Sensor Array for in Situ Measurements of Dissolved Oxygen and Redox Potential”, Environmental Science and Technology, Vol. 42, No. 22, 2007, pp. 7857-7863.

Keeping in view the above background of state of the art, it is important to note that mitochondrial dysfunctions have advanced to the forefront of clinical diagnosis in recent years. Genetic mitochondrial diseases are inherited chronic illnesses that can be present at birth or develop later. They can cause debilitating physical, developmental, and cognitive disabilities. They are progressive and there is no cure. Symptoms include poor growth, loss of muscle coordination, muscle weakness, pain and seizures. The symptoms can further include vision and/or hearing loss, gastrointestinal issues, learning disabilities, and organ failure. According to present estimates, 1 in 4,000 people has “Mito”. Mitochondrial dysfunctions, on the other hand, are associated with a broad range of chronic diseases and syndromes that impact millions of people, including diabetes and other metabolic diseases, autoimmune and inflammatory diseases, neurodegenerative diseases, and aging in general.

Preliminary understanding of mitochondrial dysfunctions and related diseases, disorders and syndromes over the last few decades have created a need for more reliable diagnostics and effective therapeutic strategies. Some attempts have been made in areas where the clinical endpoints are better understood. These include U.S. Patent Publication No. 2006/0259246 A1 to Huyn in which a biological marker identification method is proposed. The method identifies biological markers within broad sets of biological data containing many measurements. For example, the data can contain thousands of measurements on each blood sample obtained from fewer than 100 subjects, each of which falls into one or a set of clinical classes or is associated with a value of a continuous clinical response variable. At least one biomarker, containing a small subset of measurements, is found that is capable of predicting a clinical endpoint. The biomarker can be used for diagnosing disease or assessing response to a drug as an example.

Also, U.S. Patent Publication No. 2015/030105 A1 to Schettini et al. teaches that biomarkers can be assessed for diagnostics, therapy-related or prognostic methods to identify phenotypes, such as condition or disease, or the stage or progression of a disease, conditions, disease stages, and stages of a condition, and to determine treatment efficacy. The reference discloses that circulating biomarkers from a bodily fluid can be used in profiling of physiological states or determining phenotypes. The reference teaches methods of assessing microvesicles in a biological sample and an aptamer to a microvesicle surface antigen.

U.S. Patent Publication No. 2016/0223554 A1 to Cesano et al. provides an approach for the determination of the activation states of a plurality of proteins in single cells. The approach permits the rapid detection of heterogeneity in a complex cell population based on activation states, expression markers and other criteria, and the identification of cellular subsets that exhibit correlated changes in activation within the cell population. Their approach further purportedly allows the correlation of cellular activities or properties and the use of modulators of cellular activation for characterization of pathways and cell populations.

U.S. Patent Publication No. 2017/0328885 A1 to Stults et al. discloses a business method for use in classifying patient samples. The method includes steps of collecting case samples representing a clinical phenotypic state and control samples representing patients without the clinical phenotypic state. Preferably the system uses a mass spectrometry platform system to identify patterns of polypeptides in the case samples and in the control samples without regard to the specific identity of at least some of the polypeptides. Based on identified representative patterns of the state, the business method provides for the marketing of diagnostic products using representative patterns. The reference relates to systems and methods for identifying new markers, diagnosing patients with a biological state of interest, and marketing/commercializing such diagnostics of greater sensitivity, specificity, and/or cost effectiveness.

U.S. Patent Publication No. 2017/0049851 A1 to Postrel discloses improved methods for treating or preventing undesired health events including multiple related maladies, such as a disease, condition, or syndrome. The improvement results from optimization of energy metabolism by administering a therapeutically effective compound selected to a) modulate mitochondrial activity to correct for deficiencies resulting from the disease, b) boost cell energy metabolism thereby improving the original method's efficacy, and/or c) correct for metabolic disruptions resulting from therapies or medicaments used in the method to be improved. According to the reference, a combination therapy may be designed based on a disease, a group and/or an individual comprising one or more energy optimization booster combined with a medicament. Sometimes diminishing energy metabolism in selected cells to near zero may be optimal for the organism, by essentially destroying mitochondrial functionality of these cells to impair or destroy adverse functionality of these cells or subcellular activity.

U.S. Patent Publication No. 2017/0242043 A1 to Bielekova et al. describes biomarkers associated with neuro-immunological diseases. The disclosed biomarkers are secreted proteins identified in cerebral spinal fluid (CSF) samples of patients with neurological disease. The disclosed biomarkers identify patients with intrathecal inflammation, distinguish multiple sclerosis (MS) patients from patients with other types of inflammatory neurological diseases and from subjects without MS, distinguish progressive MS patients from patients with relapsing-remitting MS, identify subjects with non-MS inflammatory neurological diseases, differentiate healthy subjects from patients with any type of neurological disease, and/or identify subjects With increased disability, CNS tissue damage and/or neurodegeneration.

U.S. Pat. No. 8,645,075 B2 to Subramanian et al. teaches a systems approach based on mathematical modelling of the kinetics of essential bio-chemical pathways involved in organ homeostasis. When this in silico model is coupled with in vitro and/or in vivo measurements to quantify drug-induced perturbations, a powerful platform that allows accurate and mechanistic-level prediction of drug-induced organ injury is purported to be generated. The method described in this disclosure demonstrates that several physiological situations can also be accurately modeled in addition to the effect of perturbations induced by drugs. It can also be used along with high-throughput “-omics” data to generate testable hypotheses leading to informed decision-making in drug development.

Further in patent literature, U.S. Pat. No. 7,682,784 B2 to Kaddurah-Daouk et al. compares small molecule profiles of cells to identify small molecules which are modulated in altered states. Cellular small molecule libraries, methods of identifying tissue sources, methods for treating genetic and non-genetic diseases, and methods for predicting the efficacy of drugs are also discussed in the reference. U.S. Patent Publication Nos. 2013/0315885 A1 and U.S. Pat. No. 9,886,545 B2 to Narain et al. describe a discovery Platform Technology for analyzing a drug induced toxicity condition, such as cardiotoxicity via model building.

The Non-Patent Literature (NPL) reference entitled “Unravelling the effects of multiple experimental factors in metabolomics, analysis of human neural cells with hydrophilic interaction liquid chromatography hyphenated to high-resolution mass spectrometry”, by Victor Gonzalez-Ruiz et al. published in Journal of Chromatography A, dated Dec. 8, 2017 introduces a strategy for decomposing variable contributions within the data obtained from structured metabolomic studies. Their approach was applied in the context of an in vitro human neural model to investigate biochemical changes related to neuroinflammation. Neural cells were exposed to the neuroinflammatory toxicant trimethyltin at different doses and exposure times. In the frame of an untargeted approach, cell contents were analyzed using hydrophilic interaction chromatography (HILIC) hyphenated with high-resolution mass spectrometry (HRMS). Detected features were annotated at level 1 by comparison against a library of standards, and the 126 identified metabolites were analyzed using a recently proposed chemometric tool dedicated to multifactorial Omics datasets, namely, ANOVA multiblock orthogonal partial least squares (AMOPLS).

First, the total observed variability was decomposed to high-light the contribution of each effect related to the experimental factors. Both the dose of trimethyltin and the exposure time were found to have a statistically significant impact on the observed metabolic alterations. Cells that were exposed for a longer time exhibited a more mature and differentiated metabolome, whereas the dose of trimethyltin was linked to altered lipid pathways, which are known to participate in neurodegeneration. Then, these specific metabolic patterns were further characterized by analyzing the individual variable contributions to each effect. AMOPLS was highlighted as a useful tool for analyzing complex metabolomic data. The proposed strategy allowed the separation, quantitation and characterization of the specific contribution of the different factors and the relative importance of every metabolite to each effect with respect to the total observed variability of the system.

NPL reference entitled “Modelling of classification rules on metabolic patterns including machine learning and expert knowledge” by Christian Baumgartner et al. published in Journal of Biomedical Informatics, dated Nov. 11, 2004 teaches that machine learning has a great potential to mine potential markers from high-dimensional metabolic data without any a priori knowledge. They investigated metabolic patterns of three severe metabolic disorders, PAHD, MCADD, and 3-MCCD, on which they constructed classification models for disease screening and diagnosis using a decision tree paradigm and logistic regression analysis.

For the logistic regression analysis model-building process they assessed the relevance of established diagnostic flags, which have been developed from the biochemical knowledge of newborn metabolism and compared the models' error rates with those of the decision tree classifier. Both approaches yielded comparable classification accuracy in terms of sensitivity (>95.2%), while the LRA models built on flags showed significantly enhanced specificity. The number of false positive cases did not exceed 0.001%.

NPL reference entitled “Pattern Recognition and Classification for Multivariate Time Series” by Stephan Spiegel et al. of Technische Universitaet Berlin, dated Aug. 21, 2011 addresses the recognition of recurring patterns within multivariate time-series, which capture the evolution of multiple parameters over a certain period of time. Their approach first separates a time series into segments that can be considered as situations, and then clusters the recognized segments into groups of similar contexts. The time series segmentation is established in a bottom-up manner according the correlation of the individual signals. Recognized segments are grouped in terms of statistical features using agglomerative hierarchical clustering. The proposed approach is evaluated on the basis of real-life sensor data from different vehicles recorded during car drives. According to their evaluation it is feasible to recognize recurring patterns in time series by means of bottom-up segmentation and hierarchical clustering.

Furthermore, as attempts are also made to use machine learning techniques to predict health-related anchor measures such as ageing, disease outcomes and health risk, yet the performance of these learning models remains low. Some of these attempts can be found in “A Review of Supervised Machine Learning Applied to Ageing Research” by Fabio et al., dated 6 Mar. 2017. The reader is further referred to “A Classification Scheme for Redox-Based Modifications of Proteins” by Mark A. Perrella of Brigham and Women's Hospital, Boston, Mass., dated 2007 for additional perspective of related prevailing art.

However, even with the advanced state of the art described above, diagnostics and treatment options for mitochondrial dysfunctions and diseases is sorely lacking. What is lacking is a framework of learning systems and methods that make biomarker measurements of a broad set of analytes. It is desirable that based on these biomarker measurements the learning framework be able to identify patterns or fingerprints or signatures that are diagnostic of mitochondrial dysfunctions/diseases. Since there are over 6000 genetic diseases, many characterized by mitochondrial dysfunction, it is further desirable to develop an association or mapping of mitochondrial dysfunctions to genetic mitochondrial diseases. Such a mapping, lacking in prior art, will allow medical professionals to develop new therapeutic strategies both for primary mitochondrial diseases and diseases characterized by mitochondrial dysfunctions.

### Objects and Advantages

In view of the shortcomings of the prior art, provided herein are learning systems and methods and associated framework for deploying machine learning algorithms for learning about diseases in subjects/patients based on biomarker measurements obtained from their samples.

It is a key object of the invention to provide systems and methods for the identification and diagnosis of mitochondrial dysfunctions and associated genetic mitochondrial diseases in patients.

It is also an object of the invention to develop a library of reference bioprocess models trained or learned to predict/classify the existence of a mitochondrial dysfunction from a biomarker measurement.

Is also an object of the invention to apply its learning techniques to diagnose and treat diseases characterized by mitochondrial dysfunctions such as a neurodegenerative disease, a cardiovascular disease, a type of diabetes, a metabolic syndrome, an autoimmune disease, an inflammatory disease, a neurobehavioral disease, a psychiatric disease, a gastrointestinal disorder, a fatiguing illness, a musculoskeletal disease, a cancer, an inflammation disease and a chronic infection.

These and other objects and advantages of the invention will become apparent upon reading the detailed specification and reviewing the accompanying drawing figures.

### SUMMARY OF THE INVENTION

The present invention relates to diagnostic methods and systems that are able to diagnose and prescribe novel therapeutic strategies for genetic diseases characterized by mitochondrial dysfunctions. Such diseases are also referred to as mitochondrial diseases or genetic mitochondrial diseases. To accomplish this, a diagnostic protocol comprising a learning phase, a targeting phase and a diagnosis phase is implemented by a diagnostic platform of the present design. The diagnostic platform is computer implemented and contains computer program instructions stored in a non-transitory memory storage medium wherein the instructions are executed by one or more microprocessors to carry out the functions of the platform.

During the learning phase which is largely implemented by a learning module, a library of reference bioprocess models is developed. The models are trained on input data derived from reference biomarker measurements. A biomarker measurement is a readout or measurement of relative quantities of molecules or analytes in a sample. Such a measurement is preferably done using a high-resolution and further preferably a high-throughput mass spectrometer. As such, the readout is essentially the mass spectrum of the mass spectrometer.

The reference biomarker measurements are obtained from reference samples drawn in vitro from reference biological entities. These reference biological entities reside in respective bioreactors which may be as simple as petri dishes or any other types of bioreactors available in the art. The reference biological entities are preferably cell-cultures grown from various cell-lines. According to the main aspects, each of the cell-cultures is exposed by a suitable actuator mechanism to a chosen mitochondrial inhibitor or stressor or insult. This exposure is preferably done at the start of the stationary phase of the culture. The exposure is also preferably done in varying dosages to account for dosage-based variability in the resulting biomarker measurement. As such, there may be more than one cultures grown from the same cell-line and exposed to different doses of the mitochondrial inhibitor, inducer, stressor or insult.

Then, at varying times since the exposure, reference samples are drawn from each culture and sealed to prevent further reaction. Each of these reference samples is then analyzed by preferably a high-resolution high-throughput mass spectrometer. This analysis results in reference biomarker measurements that are essentially the mass spectra produced by the mass spectrometer per above explanation. There may be any number of cell-cultures grown from any number of cell-lines. The cell-cultures may be exposed to the mitochondrial inhibitor in any number of dosages and any number of samples may be drawn from each culture.

According to the present design, input data from these reference biomarker measurements is used to train a number of machine learning reference models. For this learning or training, various types of machine learning algorithms with various hyperparameters may be employed. In the preferred embodiments, multiple linear regression and multiple logistic regression are used. Input data is multidimensional and is conveniently represented by a 4^{th} order input tensor. The output is a vector of the predicted values. Each of the reference models is used to predict a labeled mitochondrial dysfunction induced as a result of the above-introduced mitochondrial inhibitor, stressor or insult. The above process is repeated with different mitochondrial inhibitors until a library of reference models is developed where each model is able to predict a specific labeled mitochondrial dysfunction. The models are further saved in a database for later use. The instant diagnostic platform is also able to produce a ranked list of mitochondrial dysfunctions predicted to be present in a given biological sample, in the order of probability predicted by the models.

During the targeting phase which is largely implemented by the targeting module of the diagnostic platform of the present design, target samples are obtained in vivo from a population of subjects/patients who are known to have genetic mitochondrial diseases based on their sequenced genomic data. The target samples may consist of blood or blood components, urine, stool samples, pleural fluid, ascites, sputum, tissue, plasma, tears, sweat, saliva, etc. Subsequently, target biomarker measurements are obtained from these target samples which are then analyzed by the library of reference models developed during the learning phase. If more than a statistically significant number of subjects/patients (for example, 100, 200, 500, etc.) are predicted/matched by the models to posses a labeled mitochondrial dysfunction, then a mapping/association of that mitochondrial dysfunction to the mitochondrial disease known to exist in the patients can be made.

Based on this mapping, if the matched patients who are predicted by the instant reference models to have a mitochondrial dysfunction are also known to have, based on their genetic data/patterns, the same dysfunction or a mitochondrial disease characterized by the same mitochondrial dysfunction, then this validates the instant models. Based on this mapping one can also derive insights into the correlation that exists between the original mitochondrial inhibitor and the genetic/genomic data or patterns expressed by the patients. This mapping is also stored in the database optionally along with the sequenced genomic data of the patients that was known to be causal of their genetic diseases.

Subsequently, during the diagnosis or clinical phase, which is largely implemented by a diagnosis module, a clinical sample is drawn from an undiagnosed patient. The clinical sample is any suitable sample, such as blood or blood components, urine, stool samples, pleural fluid, ascites, sputum, tissue, plasma, tears, sweat, saliva, etc. The unseen/clinical biomarker generated from this sample is then analyzed by the reference models. If the models predict a labeled mitochondrial dysfunction in the patient then based on the mapping developed in the targeting phase, the diagnosis module is able to diagnose/predict a mitochondrial dysfunction/disease in the patient, as well as the characterizing mitochondrial dysfunction and the causal inhibitor/stressor. At least, it may be used to narrow the range of potential diagnoses or to recommend a range of diagnoses for future study. The instant diagnostic platform can accomplish the above without the requirement of performing DNA sequencing on the undiagnosed patient.

Further, if the stressor has a known rescuer, then the platform may also be used to issue a therapeutic recommendation based on the rescuer, as a personalized and targeted therapy for the patient. The platform, specifically its diagnosis module, can also produce a diagnostic ranking for the patient, containing a list of potential mitochondrial dysfunctions and associated genetic mitochondrial diseases in the order of predicted probability by the reference models. The system can thus provide a prognostic forecast of the potential vulnerability or risk of the patient to those diseases in the ranking along with targeted therapeutic remedies based on any known respective rescuers.

Since mitochondrial dysfunctions are implicated in the causes of a large number of diseases, the present techniques may be employed in the diagnosis and treatment of such diseases. These diseases include at least neurodegenerative, cardiovascular, autoimmune, inflammatory, neurobehavioral, psychiatric, gastrointestinal and musculoskeletal diseases. These may also include types of diabetes, metabolic syndromes, fatiguing illnesses, cancers and chronic infections.

Consequently, a non-limiting list of neurodegenerative diseases for which mitochondrial dysfunctions may be predicted in order to improve potential treatments include Alzheimer's disease, Parkinson's disease, Huntington's disease, amyotrophic lateral sclerosis (ALS) and Friedreich's ataxia. Similarly, a non-limiting list of cardiovascular diseases for which mitochondrial dysfunctions may be predicted in order to improve potential treatments include a variety of vascular conditions including atherosclerosis. A non-limiting list of autoimmune diseases for which mitochondrial dysfunctions may be predicted in order to improve potential treatments include sclerosis, systemic lupus erythematosus and Type 1 diabetes.

In a similar manner, a non-limiting list of neurobehavioral diseases for which mitochondrial dysfunctions may be predicted in order to improve potential treatments include autism spectrum disorder, schizophrenia, a bipolar disorder, a mood disorder, depression, attention deficit hyperactivity disorder (ADHD) and post-traumatic stress disorder (PTSD). A non-limiting list of fatiguing illnesses for which mitochondrial dysfunctions may be predicted in order to improve potential treatments include chronic fatigue syndrome and Gulf War illness. A non-limiting list of musculoskeletal diseases diagnosable and treatable by the present techniques include fibromyalgia and skeletal muscle atrophy.

The diagnostic platform can employ many different learning methods using the learning framework provided herein. Some particularly useful methods in the embodiments of the present invention include Artificial Intelligence (AI) methods, Hidden Markov methods, Deep Learning (multi-layered neural network) methods or any other machine learning techniques known in the art.

The present invention, including the preferred embodiment, will now be described in detail in the below detailed description with reference to the attached drawing figures.

### BRIEF DESCRIPTION OF THE DRAWING FIGURES

FIG. 1 illustrates the diagnostic protocol according to the invention, comprising a learning phase, a targeting phase and a diagnosis phase.

FIG. 2 is a detailed system diagram illustrating its various components and interconnections for implementing the functionality of the present design.

FIG. 3 illustrates the typical lag, growth, stationary and death phases of a cell-culture.

FIG. 4 is an exemplary mass spectrum or a readout of the relative quantities of various analytes in a biomarker measurement of the present design.

FIG. 5 illustrates the “piled-up” construction of a 4^{th} order input tensor of the instant learning framework based on the multidimensional input data of the present teachings.

FIG. 6 provides a more detailed view of the diagnostic protocol and further architectural details of the diagnostic platform of the instant technology.

### DETAILED DESCRIPTION

The drawing figures and the following description relate to preferred embodiments of the present invention by way of illustration only. It should be noted that from the following discussion many alternative embodiments of the methods and systems disclosed herein will be readily recognized as viable options.

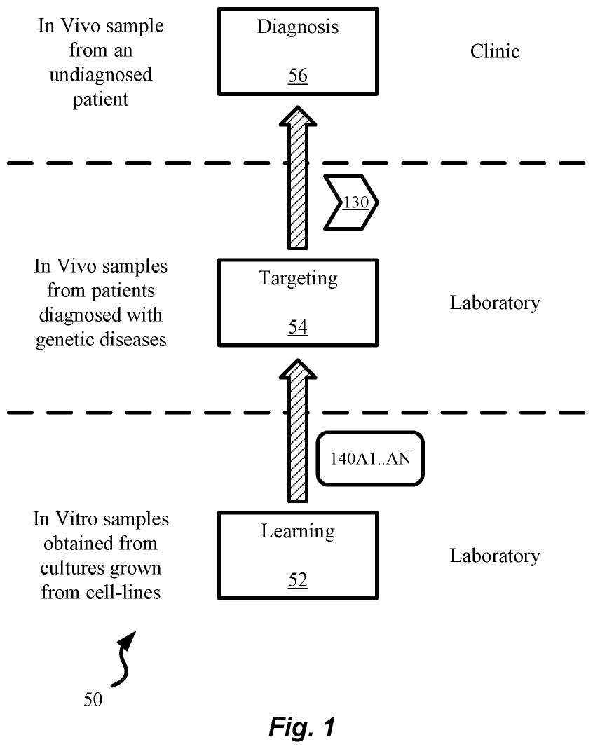

These may be employed without straying from the principles of the claimed invention. Likewise, the figures depict embodiments of the present invention for purposes of illustration only. Computer implemented learning methods and systems described herein will be best appreciated by initially reviewing the diagnostic protocol 50 as presented in FIG. 1. The diagnostic protocol of the present design has an initial learning phase as depicted by box 52. The learning phase is conducted on a number of in vitro samples that are obtained from cell-cultures grown from a number of cell-lines in a laboratory setting. As a result of this learning, a library of reference models 140A1, 140A2, . . . , 140AN are trained or learned or developed. Each of these models is able to predict a specific labeled mitochondrial dysfunction or dysfunctions given measurements of biological markers obtained from a biological sample. Each trained reference model 140A1, 140A2, . . . , 140AN is able to detect the biomarker fingerprint/signature that is indicative of a specific mitochondrial dysfunction.

The next phase of protocol 50 is referred to as targeting as shown by box 54. In targeting phase 54, models 140A1-AN are used to predict labeled mitochondrial dysfunction(s) in samples obtained from in vivo patients who have known genetic mitochondrial diseases based on genetic defects observed in their sequenced genomic data. More generally, these patients are known to have genetic diseases that are characterized by mitochondrial dysfunctions and which may or may not be expressed by the genetic information observed in the sequenced genetic data. In any case, as a resulting of targeting 54, a mapping or correspondence or association 130 of mitochondrial dysfunctions predicted by models 104A1-AN and corresponding genetic mitochondrial diseases is obtained.

Finally, in clinical, field or diagnosis phase 56 of our diagnostic protocol 50, a clinical biomarker measurement from a sample of an undiagnosed patient is used to predict the presence of a mitochondrial dysfunction(s) and any associated genetic mitochondrial diseases in that patient. The presence of any genetic diseases is determined based on mapping 130 obtained during targeting 54 and not on any genomic data obtained and sequenced from the patient. Then, based on mapping 130, diagnostic protocol 50 of the present design may be used to recommend new and personalized therapies to the patient that were heretofore unknown.

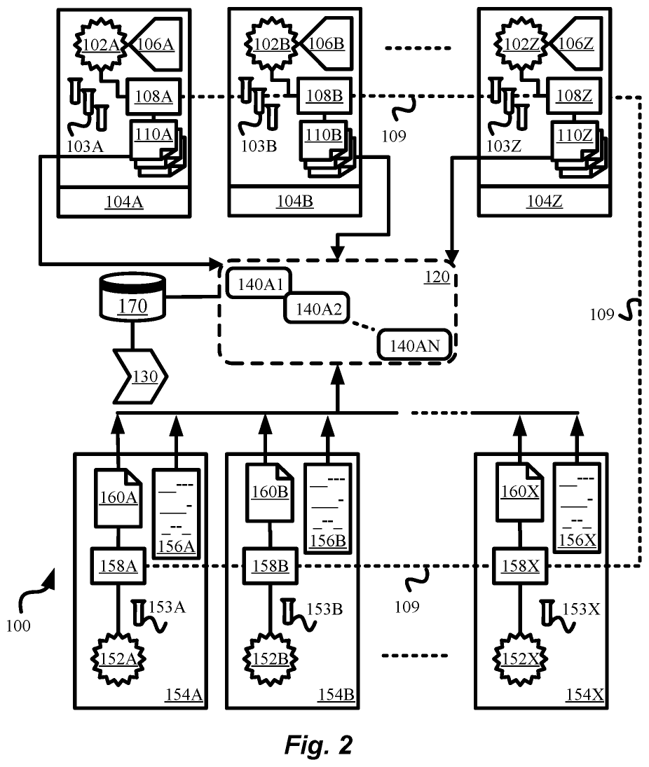

Let us now study each phase of protocol 50 of FIG. 1 in great detail. For this purpose, let us take advantage of the system diagram of the present technology as illustrated in FIG. 2. This diagram shows the key parts and interconnections of a diagnostic platform or system 100 configured to diagnose mitochondrial dysfunctions in biological processes or bioprocesses. The bioprocesses are being experienced by reference in vitro biological entities or reference in vitro cultures obtained from biological entities. Unless otherwise noted, the terms biological entities and cell-cultures that were grown from biological entities are used interchangeably in this disclosure and are referenced by numerals 102A, 102B, . . . , 102Z in FIG. 2. These biological entities or cell-cultures or simply cultures reside in respective bioreactors 104A, 104B, . . . , 104Z as shown.

It should be noted that any number of such in vitro biological cultures 102A-Z in respective bioreactors 104A-Z may be present and unless otherwise noted, the reference numerals used in this example or other examples in this disclosure are non-limiting. As such, reference numerals such as 102A-Z, 152A-X, etc. as used throughout these teachings are understood to mean any number of elements 102 rather than just 26 (A through Z) and any number of elements 152 rather than just 24 (A through X).

In the embodiment shown in FIG. 2, reference in vitro cultures 102A-Z are one or more biomasses, cell-cultures, biomaterials or biologically active substances undergoing the bioprocesses of interest as will be described below. Bioreactors 104A-Z should be understood to include dedicated reactors as well as incidental mechanisms. These include any manufactured or engineered device or system that supports a biologically active environment for growing cells or tissues, including petri dishes or cell-culture dishes. Thus, reference conditions experienced by reference in vitro cultures or biological entities 102A-Z are those existing or sustained inside bioreactors 104A-Z respectively.

At their broadest level, bioreactors 104A-Z include an engineered or managed system that supports a biologically active environment in which a chemical process is carried out that involves biological organisms or biochemically active substances or in vitro cultures 102A-Z. Bioreactors 104A-Z presented herein may range from small scale bioreactors, on the order of 10s to 100s of mL, to larger scale reactors of thousands or tens of thousands of liters. In particular, the bioreactors will typically have a volume of greater than 10 mL, greater than 100 mL, greater than 500 mL, greater than 1 L, greater than 5 L, greater than 10 L, greater than 100 L, greater than 500 L, greater than 1000 L, greater than 5000 L, greater than 10,000 L, greater than 100,000 L.

Bioprocesses of interest in the present invention involve those that include reduction-oxidation reactions. The energy involved in such a bioprocess is indicated by the voltage or potential difference ΔV equal to the redox potential Eₕ. The exact numeric value of redox potential Eₕ will depend on departure of thermodynamic conditions from standard conditions, as described by the well-known Nernst equation Eₕ=Eₒ+RT/nF·ln([A]/[B]). Here Eₒ is the standard potential for the redox couple, R is the ideal gas constant, T is the absolute temperature in degrees Kelvin, n is the number of electrons transferred in the redox reaction and F is Faraday's constant. We use the natural logarithm of the ratio of concentrations (indicated by square brackets) of the oxidized and reduced members of the redox couple A, B (e.g., NAD+ and NADH, glutathione couple GSH/GSSH or cysteine and cystine couple Cys/CySS). Those skilled in the art will also be aware of still other parameters and factors that need to be considered in assessing the redox potential of any particular redox couple (e.g., whether it is in cell, in plasma, etc.).

The redox status of a large number (e.g., hundreds or thousands) of redox couples is measurable, especially under lab conditions. On large scales, electron balance induces changes in well-known parameters, e.g., the pH value (which is a common measure of H⁺ ion concentration in moles per liter of solution expressed on a logarithmic scale). Persons skilled in the art will be very familiar with measurements of redox status using such parameters. These parameters are commonly referred to as electron balance indicators or redox indicators. Depending on conditions and available equipment, the most useful group of redox indicators can include certain oxidoreductases, oxidoreductase co-factors, electron balance influencer compounds, electron balance influencer compositions, redox-active compounds, pK values, pH values, threshold values, context measures and soft or derived indicators (usually derived with reference to a mathematical model).

FIG. 2 also shows a general apparatus used by diagnostic system 100 to learn, measure and control or adjust the redox status of the bioprocesses that reference in vitro cultures 102A, 102B, . . . , 102Z are undergoing. Respective inputs 106A, 106B, . . . , 106Z to reference bioreactors 104A-Z are provided for adjusting or altering the bioprocesses occurring inside them. Inputs 106A-Z are generally to be understood as any mechanism, actuator, inlet or other type of mechanical or non-mechanical apparatuses capable of acting on the bioprocess. Likewise, output sensors 108A, 108B, . . . , 108Z are provided for making measurements on outputs or samples 103A, 103B, . . . , 103Z drawn from the bioprocesses unfolding inside reference bioreactors 104A, 104B, . . . , 104Z respectively. These output sensors may be any type of sensing devices including mass spectrometers or even soft sensors with readings derived from various other measurements or proxies.

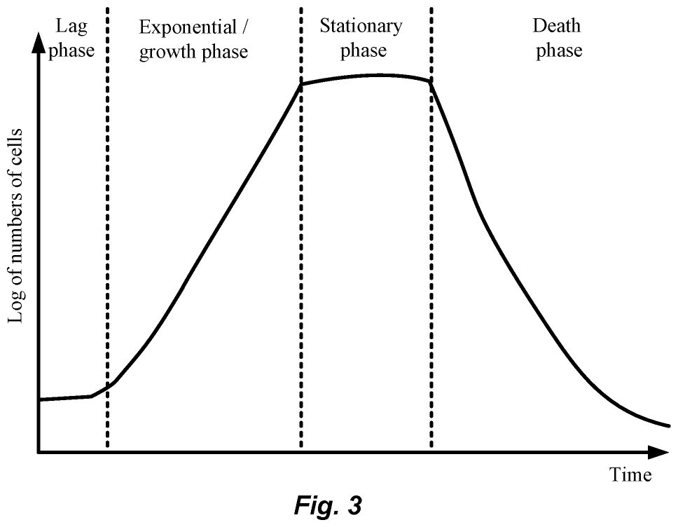

According to the main aspects of the present disclosure, a learner or data processing module 120 is used to learn any number of reference models 140A1, 140A2, . . . , 140AN. Each of models 140A1-AN is capable of predicting a mitochondrial dysfunction or dysfunctions induced in a biological entity or cell-culture due to a specific mitochondrial inhibitor, inducer, stressor or insult introduced into the entity or culture. Typically, the mitochondrial inhibitor is introduced at the beginning of the stationary phase of cell-growth of the culture although it can be done at any time during the life of the culture. This will be further discussed in reference to FIG. 3 in this disclosure.

This prediction is based on a biomarker measurement obtained from a sample drawn/taken from the entity or culture. For this purpose, learner 120 deploys one or more machine learning algorithms for learning reference models 140A1-AN. Learner 120 runs on a dedicated computer, computer system or even a computer cluster that is collocated or geographically distributed (not shown). A person skilled in the art will appreciate, that many types of resources and architectures can support the execution of data processing module or learner 120. Furthermore, processing module 120 is understood to execute program instructions by one or more processors in order to carry out its functions as described herein. The program instructions are stored in one or more non-transitory storage media that is/are coupled to the one or more microprocessors.

Output sensors 108A-Z perform reference measurements on reference in vitro samples taken from in vitro cultures 102A-Z. The reference in vitro samples taken for measurements are shown by vials of which only one is marked by reference numerals 103A, 103B, . . . , 103Z to avoid clutter. In practice, instead of or in addition to vials, any other mechanical or non-mechanical biological sampling mechanisms including tubing, suction and other techniques may also be used to extract samples 103A-Z.

Once a sample is drawn/extracted at a given instant of time, it is sealed to stop further reaction and oxidation. A sample, for example one of samples 103B, is thus representative of the stage or the moment in time of the reaction of culture 102B at which that specific sample 103B was drawn. Recall from above that reference numeral 103B may represent more than one samples extracted from culture 102B. The techniques for drawing/extracting samples 103A-Z from cell-cultures or biological entities 102A-Z are known in the art and will not be delved into detail in this disclosure.

The reference measurements result in respective biomarker measurements 110A-Z of reference in vitro cultures 102A-Z, each measurement corresponding to an instant of time or a stage of the reaction of the culture at which the respective sample 103A-Z was drawn. Thus, each of in vitro samples 103A will result in a reference biomarker measurement, however only one such biomarker measurement is marked with reference numeral 110A for reasons of clarity. For the same reasons, only one of biomarker measurement obtained from samples 103B is marked with 110B and so on. Reference biomarker measurements 110A-Z contain measured quantities of analytes belonging to several categories of redox data based on the redox code. The measurements may contain analytes or metabolites belonging to the various metabolomes related to the bioprocesses which cultures 102A-Z are undergoing.

The redox code includes the four principles by which biological systems are organized. The first category contains bio-energetics redox data. These are data pertaining to catabolism and anabolism typically organized through high-flux NAD and NADP systems. The second category contains macromolecular structure and activities that are linked to bio-energetic systems through kinetically controlled sulfur switches. This category is referred to as switching redox data. The third category contains signaling redox data. This category relates to activation and deactivation cycles, e.g., of H₂O₂ production (usually linked to NAD and NADP systems to support redox signaling and spatiotemporal sequencing for differentiation and multicellular development). The fourth category contains network redox data. This type of data relates to redox networks, from micro-compartments to subcellular and cellular organization and includes adaptive responses to the environment.

In addition to the four redox code categories, reference biomarker measurements 110A-Z may also contain a fifth category of data. This fifth category includes contingent redox data. Contingent redox data includes candidates (e.g., candidate redox indicators that are speculative) for any of the first four categories, as well as contextual information having to do with reference conditions or environment in which reference bioprocess of in vitro cell-cultures 102A-Z transpire. Contingent data can also include other types of information that may be relevant directly or indirectly to oxidation-reduction activity or charge balance. It is possible for contingent redox data to encompass contextual information that can only be inferred from factors not specifically related in any known way to charge balance. Contingent redox data can also include common annotations, labels and other information that curators or experts typically add to ensure proper understanding of the data.

Reference biomarker measurements 110A-Z can also include information that is not directly measurable, also known herein as “soft data”. Such “soft data” is inferred on a model applied to a collection of surrogate measures that are weighted to estimate or infer a measure of interest. For more information about soft sensors and soft data the reader is referred to Paulsson D., et al., “A Sensor for Bioprocess Control Based on Sequential Filtering of Metabolic Heat Signals”, Vol. 14, Sensors, 26 Sep. 2014, pp. 17864-17882.

Processing module 120 is configured to receive reference biomarker measurements 110A-Z from reference in vitro samples taken from respective in vitro biological entities or cell-cultures 102A-Z grown in the lab. In the event that biological entities or in vitro cultures 102A-Z undergoing the bioprocesses in reference bioreactors 104A-Z require frequent or even continuous monitoring, the delay in the communication of reference biomarker measurements 110A-Z to learner 120 should be kept as short as practicable. In such cases, geographic collocation of the computer(s) running processing module 120 with bioreactors 104A-Z containing in vitro samples 102A-Z is preferred. A person skilled in the art will be able to make the appropriate decision about the distribution and assignment of the correspondent computational tasks and in vitro samples 102A-Z in the lab conditions.

In accordance with the instant design, processing module 120 deploys one or more machine learning algorithms for learning from reference biomarker measurements 110A-Z, reference models 140A1-AN. The learning algorithms may operate in supervised or unsupervised mode. After the learning or training, each reference model 140A1-AN is able to predict a labeled mitochondrial dysfunction present in any biological sample based on its biomarker fingerprint detected in input biomarker measurements 110A-Z. In other words, the biomarker fingerprint itself is the specific subset or pattern or principal components/weights from all measurements 110A-Z in the lab which most successfully predicts D1. We also at times refer to such a fingerprint as conserved enough across the runs to be able to predict D1.

In this disclosure, the set of dysfunction/dysfunctions thus predicted are identified by a label, such as “D1”, “D2”, etc. each associated with the mitochondrial inhibitor, stress or insult known to induce the mitochondrial dysfunction. In other words, the training data for the machine learning process is labeled by the known dysfunction associated with the sample from which the data was generated. More specifically, data processing module or processing module or learning module or simply learner 120 trains a number of machine learning models 140A1-N that predict the presence of a certain mitochondrial dysfunction/dysfunctions present in the biological sample from which the biomarker measurement was obtained. This learning process will be taught in much more detail further below.

Learner 120 learns reference models 140A1-AN on training data based on reference biomarker measurements obtained from in vitro samples drawn from in vitro cell-cultures 102A-Z that are perturbed by respective inputs or actuator mechanisms 106A-Z operating on them. Actuator systems 106A, 106B, . . . , 106Z interface with respective bioreactors 104A, 104B, . . . , 104Z. Each of actuators 106A-Z deploys one or more individual input mechanisms to control, provide inputs to, or in any other way, alter or perturb or adjust the bioprocess transpiring in the respective reference biological entity or reference in vitro culture 102A-Z housed in respective bioreactor 102A-Z.

It is noted that in some embodiments any of actuators 106A-Z may only utilize on or more actuator or input mechanisms, e.g., a stirrer or just an inlet pipe or multiple inputs or inlet pipes, coupled to multiple sources of inputs to supply additional quantities of culture material to in vitro cultures 102A-Z, or to provide still any other input material. These other inputs could include other feed stock or biomaterials, including, e.g., redox influencers or mitochondrial inhibitors, inducers, stressors or insults. Further, the inputs may also include: off-gas, air, O₂, CO₂, pressure, viscosity, stirrer speed, temperature, pO₂, pH, photometrics, calorespirometric measures and other biomeasureables. Of course, there may be cases in which control of the local bioprocess is impossible or impractical. This could occur in rapidly transpiring reactions or reactions that go to completion without allowing for meaningful intervention. Actuator system 106 can also recommend an operation to a local operator (not shown).

Specific bioprocesses transpiring in each reference biological entity or in vitro culture 102A, 102B, . . . , 102Z are sensed or measured at various stages by first drawing in vitro samples 103A-Z from the cultures and then monitoring and/or measuring them by corresponding sensor systems 108A, 108B, . . . , 108Z. Although not explicitly shown, each sensor system 108A, 108B, . . . , 108Z may include one or more individual measurement devices, sensors and/or monitors as well as any requisite interfaces, hardware and software.

Sensors 108A-Z can also include high-resolution and high-throughput mass spectrometers. Reference biomarker measurements 110A-Z can take into account mass spectrometer results resolving as many as 20,000 or even 50,000 or more potential peaks to locate known or targeted redox indicators for the bioprocesses of in vitro samples 102A-Z. Alternatively, if sufficient processing power is employed, a full or partially untargeted set of peaks may be measured to associate the mitochondrial dysfunction label with patterns of analytes without any previous knowledge about the particular mechanisms of the analytes.

The above is advantageously accomplished by using a high-resolution mass spectrometer in which mass-to-charge ratio (m/z) for each ion is measured to several decimal places to differentiate between molecular formulas having similar masses. Potential mass spectrometers include instruments supplied by commercial manufacturers such as AB Sciex, Advion, Agilent, Applied biosystems, Bruker, GenTech Scientific, Hitachi High Technologies, IONICON, JEOL, LECO, PerkinElmer, Shimadzu, Thermo Fisher Scientific, Waters and others.

Measurements 110A-Z used to train each of reference models 140A1-AN to predict a labeled mitochondrial dysfunction can comprise a range of mass spectrometry outputs/peaks, any other sensor data for specific analyte measurements or even the conditions of bioreactors 104A-Z, any soft sensors of the bioreactors, or still any other type of measurement data. Respective inputs/actuators 106A-Z can be used and other settings or environmental conditions of the bioreactors can be varied to create more data sets so that the models predicting the mitochondrial dysfunctions are more stable over a broad range of conditions. This way, the models are not over-trained on data/conditions not conserved across the wide variety of conditions/situations characteristic of mitochondrial dysfunction diagnostics under in vivo conditions.

In many practical situations, a mass spectrometer is a valuable and shared resource, so it will be prudent that in vitro samples 103A-Z taken from multiple respective biological entities or cultures 102A-Z are measured by a common mass spectrometer. As such, mass spectrometer or sensor 108 will be a device common for measuring the outputs of the one or more cultures. This sharing of the same sensor/spectrometer 108 in FIG. 2 is illustrated by dotted line 109. For this reason, it is also desirable to have sensor/spectrometer 108 that is high-throughput and is able to process a large volume of samples at preferably high-resolution with efficiency.

Diagnostic platform 100 of FIG. 2 is understood to include the requisite control interfaces operatively coupled to actuators 106A-Z as well as the requisite processors and/or control interfaces coupled to sensors 108A-Z. These processors and/or control interfaces may be instructed and supervised by processing module 120 according to the techniques provided herein. Alternatively, they may be controlled by other external systems and devices not specifically shown in FIG. 2.

For example, once samples 103A-Z are extracted from cultures 102A-Z respectively at the desired times, respective sensors 108A-Z may obtain biomarker measurements at their own behest or under the supervision of an external module and provide or “push” the corresponding data in the form of respective measurements 110A-Z to processing module 120. Alternatively, processing module 120 may instruct sensors 108A-Z to perform their reference measurements and subsequently “pull” the measurement data in the form of respective biomarker measurements 110A-Z.

In the preferred embodiment, each of in vitro cultures 102A-Z is grown in the lab from a specific cell-line. In a given “run” or a set of experiments, there may be several cultures of a given cell-line present. Then, reference actuator mechanisms 106A-Z introduce a given/selected mitochondrial inhibitor/inducers/stressor/insult into each of in vitro cultures 102. This introduction is preferably performed at the start of the stationary phase of cell-growth of the respective cultures, or simply stated the stationary phase of the respective cultures.

Furthermore, the introduction is preferably carried out in varying dosages. In other words, the cultures from a given cell-line may each be given a varying dosage of the mitochondrial inhibitor so that their biomarker measurements may be made under different exposure concentrations.

One of the dosage amounts needs to be 0/zero or in other words, no introduction of the mitochondrial inhibitor. This 0/zero state is necessary because the learning process needs to recognize those patterns that are just generated by a given in vitro culture in its natural or uninhibited state. It will then need to eliminate those patterns when predicting mitochondrial dysfunction in the culture in when it is in its perturbed state.

Alternatively, the zero state may be thought of as the labeled state defined by a null hypothesis inhibitor. In such a scenario, learning module/engine 120 can be used to train a model that predicts the zero state just as it can train a model to predict the labeled mitochondrial dysfunction caused by a specific mitochondrial inhibitor. In either case, the label corresponds to a state of mitochondrial function/dysfunction that is measured in biomarker measurements 110A-Z in vitro. The learning process will be explained in detail further below.

Reference measurements are also preferably performed a number of times in a time series manner by sensor mechanisms 108A-Z on respective reference in vitro cultures 102A-Z. Explained further, each of these reference measurements are performed on respective in vitro samples 103A-Z drawn from cultures 102A-Z at times t₁, t₂, . . . , tᵢ. Typically, times t₁, t₂, . . . , tᵢ are measured as time intervals since the exposure of the culture to the mitochondrial inhibitor chosen for the runs. Recall that the culture is preferably exposed to the mitochondrial inhibitor at the start of the stationary phase of the cell-growth of the culture, although this exposure may be done at any time during the life of the culture.

Thus, reference numeral 103A refers to one or more samples drawn from culture 102A at varying times t₁, t₂, . . . , tᵢ since the exposure/perturbation of the culture with the specific mitochondrial inhibitor. Similarly, reference numeral 103B refers to one or more samples drawn from culture 102B at varying times t₁, t₂, . . . , tᵢ since the exposure/perturbation of the culture with the same specific mitochondrial inhibitor, and so on. In some embodiments, samples 103 may also be drawn from cultures 102 under differing environmental conditions that may influence the cell culture, such as temperature, pressure, exposure to gases, etc. Such differing conditions may be effected by actuators/inputs 106 as noted above.

A measurement of biomarkers or analytes from a given in vitro sample is referred to as a reference biomarker measurement 110. Thus, reference numeral 110A refers to one or more reference biomarker measurements from respective samples 103A at varying times t₁, t₂, . . . , tᵢ since the exposure/perturbation of culture 102A with the mitochondrial inhibitor and under varying conditions. Similarly, reference numeral 110B refers to one or more reference biomarker measurements from respective samples 103B at varying times t₁, t₂, . . . , tᵢ since the exposure/perturbation of culture 102B with the mitochondrial inhibitor and under varying conditions, and so on. Note that there is no requirement that the number of samples 103A-Z drawn from respective cultures 102A-Z resulting in corresponding biomarker measurements 110A-Z be all the same in number. The equal number (3) of vials of samples 103A, 103B, . . . , 103Z and biomarker measurements 110A, 110B, . . . , 110Z shown in FIG. 2 is for exemplary purposes only.

For a set of runs involving a given mitochondrial inhibitor applied to a given number of cell-cultures grown from a given number of cell-lines, the total number of reference measurements will be:

(1 mitochondrial inhibitor+1 (when no mitochondrial inhibitor is used))×number of cell-cultures grown×number of cell-lines×i (number of time series measurements)).

Each of these reference measurements is measured by sensor 108A-Z, such as a high-resolution, high-throughput mass spectrometer.

In a similar manner, another set of reference runs or experiments is conducted for a different mitochondrial inhibitor, and so on for other mitochondrial inhibitors.

The total number of reference in vitro measurements are thus:

(Total number of mitochondrial inhibitors+1)×Total number of cell-cultures from each cell-line×Total number of cell-lines×Sampling rate i or the total number of times at which the measurements are taken.

In an exemplary scenario, with 13 inhibitors and 20 cell-cultures from 97 cell-lines and with 20 time-based measurements, the total number of reference biomarker measurements are: 14×97×20×20=543200.

Table 1 lists exemplary neurobiology cell-lines that may be used for growing in vitro cell-cultures 102A-Z while Table 2 lists exemplary mitochondrial inhibitors, both provided by Sigma-Aldrich. Thus, exemplary inhibitors/inducers/stressors/insults in Table 2 may be used as perturbations for exemplary cultures derived from Table 1 in the above described reference runs/experiments.

TABLE 1

| No. | Cell Name | Description |
|---|---|---|
| 1 | DI TNC1 | Rat Astrocyte transfected |
| 2 | CTX TNA2 | Rat Astrocyte, Transfected |
| 3 | BE(2)-C | Human Caucasian neuroblastoma |
| 4 | BE(2)-M17 | Human Caucasian neuroblastoma |
| 5 | SK-N-BE(2) | Human Caucasian neuroblastoma |
| 6 | SK-N-DZ | Human neuroblastoma |
| 7 | SH-SY5Y | Human neuroblastoma |
| 8 | C1300 | Mouse neuroblastoma |
| | CLONE NA | |
| 9 | ND-C | Mouse neuroblastoma × Rat neurone |
| | | hybrid |
| 10 | ND27 | Mouse neuroblastoma × Rat neurone |
| | | hybrid |
| 11 | ND15 | Mouse neuroblastoma × Rat neurone |
| | | hybrid |
| 12 | ND8/34 | Mouse neuroblastoma × Rat neurone |
| | | hybrid |
| 13 | ND7/23 | Mouse neuroblastoma × Rat neurone |
| | | hybrid |
| 14 | ND3 | Mouse neuroblastoma × Rat neurone |
| | | hybrid |
| 15 | C6 | Rat glial tumour |
| 16 | NB4 1A3 | Mouse C-1300 Neuroblastoma |
| 17 | Neuro 2a | Mouse Albino neuroblastoma |
| 18 | N1E-115 | Mouse neuroblastoma |
| 19 | NG108-15 | Mouse neuroblastoma × Rat glioma |
| | | hybrid |
| 20 | N18 | Mouse neuroblastoma × Rat glioma |
| | | hybrid |
| 21 | 33B | Rat nervous tissue |
| | | oligodendroglioma |
| 22 | B65 | Rat nervous tissue neuronal |
| 23 | B92 | Rat nervous tissue glial |
| 24 | B50 | Rat nervous tissue neuronal |
| 25 | CAD | Mouse (B6/D2 F1 hybrid) |
| | | catecholaminergic neuronal tumour |
| 26 | F11 | Rat embryonic dorsal root |
| | | ganglion |
| 27 | 108CC5T-BU-4 | Mouse neuroblastoma × Rat glioma |
| | | hybrid |
| 28 | 108CC5T-BU-1 | Mouse neuroblastoma × Rat glioma |
| | | hybrid |
| 29 | 108CC5-BU-8 | Mouse neuroblastoma × Rat glioma |
| | | hybrid |
| 30 | 108CC5-BU-5 | Mouse neuroblastoma × Rat glioma |
| | | hybrid |
| 31 | 108CC5-BU | Mouse neuroblastoma × Rat glioma |
| | | hybrid |
| 32 | N115-BU-10 | Mouse Neuroblastoma |
| 33 | N115-BU-9 | Mouse Neuroblastoma |
| 34 | N115-BU-7 | Mouse Neuroblastoma |
| 35 | N115-BU-2 | Mouse neuroblastoma, |
| | | bromodeoxyuridine resistant |
| 36 | 108CC5-TG-4 | Mouse neuroblastoma × Rat glioma |
| | | hybrid |
| 37 | 108CC5-TG-3 | Mouse neuroblastoma × Rat glioma |
| | | hybrid |
| 38 | 108CC5-TG-2 | Mouse neuroblastoma × Rat glioma |
| | | hybrid |
| 39 | 108CC5-TG-1 | Mouse neuroblastoma × Rat glioma |
| | | hybrid |
| 40 | 328/14 | Mouse neuroblastoma × mouse L |
| | | cell fibroblast hybrid |
| 41 | 328/12 | Mouse neuroblastoma × mouse L |
| | | cell fibroblast hybrid |
| 42 | 328/11 | Mouse neuroblastoma × mouse L |
| | | cell fibroblast hybrid |
| 43 | 328/10 | Mouse neuroblastoma × mouse L |
| | | cell fibroblast hybrid |
| 44 | 328/9 | Mouse neuroblastoma × mouse L |
| | | cell fibroblast hybrid |
| 45 | 328/8 | Mouse neuroblastoma × mouse L |
| | | cell fibroblast hybrid |
| 46 | 328/7 | Mouse neuroblastoma × mouse L |
| | | cell fibroblast hybrid |
| 47 | NH15-CA2 | Mouse neuroblastoma × Rat glioma |
| | | hybrid |
| 48 | N18TG2 | Mouse neuroblastoma |
| 49 | 108CC5 | Mouse neuroblastoma × Rat glioma |
| | | hybrid |
| 50 | N115-BU-8 | Mouse Neuroblastoma |
| 51 | NS20Y | Mouse neuroblastoma |
| 52 | 108CC15 | Mouse neuroblastoma × Rat glioma |
| | | hybrid |
| 53 | N4TG3 | Mouse neuroblastoma |
| 54 | NS20Y-TG | Mouse neuroblastoma |
| 55 | N1E-115-1 | Mouse neuroblastoma |
| 56 | NS20Y-BU-7 | Mouse neuroblastoma |
| 57 | NS20Y-BU-6 | Mouse neuroblastoma |
| 58 | NS20Y-BU-5 | Mouse neuroblastoma |
| 59 | NS20Y-BU-4 | Mouse neuroblastoma |
| 60 | NS20Y-BU-2 | Mouse neuroblastoma |
| 61 | C17.2 | Mouse multipotent neural |
| | | progenitor or stem-like cells |
| 62 | CHP-134 | Human neuroblastoma tumour mass |
| | | of left adrenal gland |
| 63 | LAl-5s | Human Neural Crest-Derived Non- |
| | | Neuronal Progenitor |
| 64 | LA-N-1 | Human Neuroblastoma Bone Marrow |
| | | Metastasis |
| 65 | WERI | Human Retinoblastoma |
| 66 | Rolf B1.T | Adult rat olfactory nerve |
| | | ensheathing cells |
| 67 | Y79 | Human Caucasian retinoblastoma |
| 68 | RB247C | Human Retinoblastoma |
| 69 | 1321N1 | Human brain astrocytoma |
| 70 | A15 | Rat, BDIX, glioma |
| 71 | ANGM-CSS | Human glioblastoma |
| 72 | b.End3 | Mouse SV129 brain endothelioma |
| 73 | b.End5 | Mouse Balb/c brain endothelioma |
| 74 | BC3H1 | Mouse brain tumour |
| 75 | BE10-7 | Rat, BDIX, brain, pre-malignant |
| 76 | BE10- | Rat, BDIX, foetal brain, pre- |
| | Intermediate | malignant |
| 77 | BE10-Late | Rat, BDIX, foetal brain, |
| | | malignant |
| 78 | BE11 | Rat, BDIX, foetal brain |
| | (Early) | |
| 79 | C6-2-3 | Rat glioma × rat glioma hybrid |
| 80 | C6-4-2 | Rat glioma × rat glioma hybrid |
| 81 | C6-BU-1 | Rat glioma × rat glioma hybrid |
| 82 | CCF-STTG1 | Human Caucasian astrocytoma |
| 83 | DBTRG.05MG | Human glioblastoma |
| 84 | GPNT | An immortalised Lewis rat brain |
| | | vascular endothelial cell-line |
| 85 | KELLY | Human neuroblastoma |
| 86 | MOG-G-CCM | Human brain astrocytoma |
| 87 | MOG-G-UVW | Human brain astrocytoma |
| 88 | NB69 | Human neuroblastoma (Stage III) |
| 89 | PG-4 | Cat brain Moloney sarcoma virus- |
| | | transformed |
| 90 | SCP | Ovine brain choroid plexus |
| 91 | T98G | Human Caucasian glioblastoma |
| 92 | TR33B | Rat Wistar-Furth |
| | | oligodendroglioma |
| 93 | U-87 MG | Human glioblastoma astrocytoma |
| 94 | U-251 MG | Human glioblastoma astrocytoma |
| | (formerly | |
| | known as | |
| | U-373 MG) | |
| 95 | U-373 MG | Human glioblastoma astrocytoma |
| | (Uppsala) | |
| 96 | RN46A-B14 | Embryonic rat medullary raphe, |
| | | temperature-sensitive mutant of |
| | | SV40 large T-antigen, |
| | | immortalised, serotonergic, |
| | | neuronal |
| 97 | RN4 6A | Embryonic rat medullary raphe, |
| | | temperature-sensitive mutant of |
| | | SV40 large T-antigen, |
| | | immortalised, serotonergic, |
| | | neuronal |

TABLE 2

| | Mitochondrial | | Mitochondrial dysfunction |
|---|---|---|---|
| Label | Inhibitor | Description | induced |
| D1 | A8674 | Antimycin A | Inhibits electron transfer at |
| | | from | complex III. Induces apoptosis. |
| | | Streptomyces sp. | |
| D2 | BM0017 | BMS-199264 | Potently inhibits the ATP |
| | | hydrochloride ≥ 98% | hydrolase activity of |
| | | (HPLC) | mitochondrial FIFO ATP |
| | | | synthase. The compound BMS- |
| | | | 199624 has no effect on the ATP |
| | | | synthase function of FIFO. In |
| | | | isolated rat hearts, BMS-199624 |
| | | | blocks depletion of ATP levels, |
| | | | and blocks necrosis during |
| | | | ischemia. |
| D3 | SML1122 | BTB06584 ≥ 98% | BTB06584 inhibits the ATP |
| | | (HPLC) | hydrolase activity of |
| | | | mitochondrial FIFO ATP |
| | | | synthase. The compound BTB06584 |
| | | | has no effect on oxygen |
| | | | consumption or mitochondrial |
| | | | membrane potential in HL-1, a |
| | | | mouse cardiac cell-line, but |
| | | | blocks ATP consumption and |
| | | | ischemic cell death following |
| | | | inhibition of cellular |
| | | | respiration. |
| D4 | C2759 | Carbonyl | Protonophore (H+ ionophore) and |
| | | cyanide 3- | uncoupler of oxidative |
| | | chlorophenyl- | phosphorylation in |
| | | hydrazone ≥ 97% | mitochondria. Shown to have a |
| | | (TLC), powder | number of effects on cellular |
| | | | calcium. Inhibits secretion of |
| | | | hepatic lipase and partially |
| | | | inhibits the pH gradient- |
| | | | activated Cl- uptake and Cl-/ |
| | | | Cl- exchange activities in |
| | | | brush-border membrane vesicles. |
| D5 | C2920 | Carbonyl | FCCP is a protonophore (H+ |
| | | cyanide 4- | ionophore) and uncoupler of |
| | | (trifluoro- | oxidative phosphorylation in |
| | | methoxy)phenyl- | mitochondria. It is capable of |
| | | hydrazone ≥ 98% | depolarizing plasma and |
| | | (TLC), powder | mitochondrial membranes. FCCP |
| | | | has been shown to have a number |
| | | | of effects on cellular calcium. |
| | | | It also is reported to inhibit |
| | | | a background K+ current and |
| | | | induce a small inward current, |
| | | | reduce pH by 0.1 unit, and |
| | | | induce a rise of intracellular |
| | | | [Na+]. FCCP stimulates Mg2+- |
| | | | ATPase activity, inhibits β- |
| | | | amyloid production, and mimics |
| | | | the effect of selective |
| | | | glutamate agonist N-methyl-D- |
| | | | aspartate (NMDA) on |
| | | | mitochondrial superoxide |
| | | | production. |
| D6 | C2020 | α-Cyano-4- | Specific inhibitor of |
| | | hydroxycinnamic | monocarboxylic acid transport, |
| | | acid ≥ 98% | including lactate and pyruvate |
| | | (TLC), powder | transport. Also reported to |
| | | | block β-cell apical anion |
| | | | exchange (IC50 of 2.4 mM). |
| D7 | I9890 | m-Iodobenzyl-guanidine | Antitumor agent which inhibits |
| | | hemisulfate | ADP ribosylation. |
| | | salt ≥ 98% | |
| | | (HPLC and | |
| | | TLC) | |
| D8 | L4900 | Lonidamine | Inhibits the energy metabolism |
| | | mitochondrial | of neoplastic cells by |
| | | hexokinase | interfering with hexokinase and |
| | | inhibitor | disrupting uncoupler-stimulated |
| | | | mitochondrial electron |
| | | | transport; damages cell and |
| | | | mitochondrial membranes. |
| D9 | M2324 | ML-3H2 | ML-3H2 is an allosteric |
| | | | hexamer-stabilizing inhibitor |
| | | | of human porphobilinogen |
| | | | synthase (PBGS; ALAD) |
| D10 | 04876 | Oligomycin | Macrolide antibiotic; inhibits |
| | | from | mitochondrial ATPase and |
| | | Streptomyces | phosphoryl group transfer. |
| | | diastato- | |
| | | chromogenes ≥ 90% | |
| | | total | |
| | | oligomycins | |
| | | basis (HPLC) | |
| D11 | P8861 | Pyrrolnitrin | Pyrrolnitrin blocks the |
| | | from | terminal electron transport |
| | | Pseudomonas | between succinate or reduced |
| | | cepacia ≥ 98% | NADH and coenzyme Q. In |
| | | (HPLC), solid | mitochondria preparations of S. |
| | | | cerevisiae, the antibiotic |
| | | | inhibited succinate oxidase, |
| | | | NADH oxidase, succinate |
| | | | cythochrome C reductase, and |
| | | | NADH-cytochrome C reductase. |
| | | | Pyrrolnitrin is involved in |
| | | | many cellular processes such as |
| | | | oxidative stress, electron |
| | | | transport, DNA and RNA |
| | | | synthesis. |
| D12 | R8875 | Rotenone ≥ 95% | Inhibitor of mitochondrial |
| | | | electron transport at |
| | | | NADH:ubiquinone oxidoreductase. |
| | | | It is readily absorbed through |
| | | | the exoskeletons of arthropods, |
| | | | but poorly absorbed cutaneously |
| | | | or from the gastrointestinal |
| | | | tract of mammals. Rotenone is |
| | | | used to induce a Parkinson-like |
| | | | syndrome as an experimental |
| | | | model in rats. |
| | | | Inhibitor of mitochondrial |
| | | | electron transport. Neurotoxic |
| | | | agent that can produce a |
| | | | Parkinson-like condition as an |
| | | | animal model for study of |
| | | | etiology and interventions. |
| D13 | SML1280 | TT01001 ≥ 98% | TT01001 is potent, orally |
| | | (HPLC) | available the mitochondrial |
| | | | outer membrane protein mitoNEET |
| | | | ligands that binds to mitoNEET |
| | | | without PPARγ activation. |
| | | | TT01001 improves hyperglycemia, |
| | | | hyperlipidemia, and glucose |
| | | | intolerance in mice models of |
| | | | diabetes II. TT01001 exerts |
| | | | anti-diabetic effects without |
| | | | the pioglitazone associated |
| | | | weight gain. |

Each biomarker measurement 110A-Z consists of the measurements indicating the presence and concentrations of a variety of analytes observed in the sample by an appropriate sensor, such as a mass spectrometer. In this disclosure, analytes refer to any element/compound such as a metabolite or a redox indicator and/or its cofactor discussed above, that is observed by a measuring instrument such as a spectrometer. As also noted above, redox balance is due to relative oxidation/reduction status between redox couples operating at the physical chemistry level. Some of the suitable couples without their co-factors are listed in Tables 3, 4 and 5 below. More precisely, Tables 3-5 provide an exemplary and partial list of analytes that are measured in the biomarker measurements of the instant teachings.

TABLE 3
Redox Pairs

| | Analytes |
|---|---|
| Panel 1 | Cystine* |
| | Cysteine* |
| | Cysteine Persulfide* |
| | GSSG* |
| | GSH* |
| | GSH Persulfide* |
| | HomoCystine* |
| | XOMA |
| | H₂S* |
| | Thiosulfate* |
| | Tetrathionate |
| | CysGly Dipeptide* |
| | GluCys Dipeptide* |
| | Cys-GSH Disulfide |
| | Ophthalmic Acid* |
| | Cystathionine |
| | Lanthionine |
| | GSH-Sulfonic Acid |
| | Lipoic Acid |
| | Cysteamine |
| | Methionine* |
| | Adenosine* |
| | SAM* |
| | SAH |
| | Spermine* |
| | Spermidine* |
| | Citrulline* |
| | Ornithine |
| | Kynurenine |
| | Kynurenic Acid |
| | Serine |
| | Taurine* |
| | Pyroglutamic Acid |
| | α-Aminobutyric Acid* |
| | 3-NitroTyrosine* |
| | 3-ChloroTyrosine* |
| | Glutamate |
| | Homocitrilline |
| | Aspartate |

*Isotopically Labeled Standard used

TABLE 4
Redox Pairs

| | Analytes |
|---|---|
| Panel 2 | NAD |
| | NADP |
| | AMP |
| | ADP |
| | ATP |
| | CAMP |
| | Xanthine |
| | Hypoxanthine* |
| | 2-deoxy-guanosine* |
| | Inosine |
| | Acetyl-Carnitine* |
| | Carnitine |
| | NADH |
| | NADPH |
| | Urate |
| | 8-OH-dG |
| | Pyrimido purinone |
| | Fumurate* |
| | Succinate* |
| | Lactate* |
| | Pyruvate* |
| | Acetoacetate |
| | 3-Hydroxybutyric Acid |
| | 743-OH |
| | 743* |
| | 886 |
| | A0001-OH |
| | A0001* |
| | α-TOC |
| | α-CEHC |
| | δ-CEHC |
| | 743-OH-Sulfate |
| | 743-OH-Gluc |
| | A0001-OH-Sulfate |
| | A0001-OH-Gluc |
| | 589* |
| | 589-OH |
| | 589-Sulfate |
| | 589-Gluc |

*Isotopically Labeled Standard used

TABLE 5
Redox Pairs

| | Analytes |
|---|---|
| Panel 3 | CoQ10 |
| | Ubiquinol (CoQ10-OH) |
| | Docosahexaenoic Acid (DHA)* |
| | Arachidonic Acid (AA)* |
| | Linoleic Acid |
| | Palmitoyl Carnitine |
| | Prostaglandin E2* |
| | tetranor PGE-M* |
| | tetranor PGA-M |
| | 15-Deoxy-PGJ2 |
| | 15-Deoxy-PGJ2-GSH |
| | Leukotriene E4* |
| | Leukotriene C4 |
| | 8-iso-PGF2a* |
| | Creatinine (urine) |
| | 2,3-DPG (RBC contamination of plasma) |

*Isotopically Labeled Standard used

Particularly useful analytes measured by sensor mechanisms 108 include the presence or concentration of an oxidoreductase or of an oxidoreductase co-factor. Others include the presence or concentration of balance influencer compounds, electron balance influencer compositions or still other redox-active compounds. Tables 3-5 above provide only a partial list of such analytes that are measured by sensor mechanisms 108 of FIG. 2. A full list of such measurable analytes includes tens of thousands or more compounds and will be accessible to a person of average skill. Still other analytes of interest for measurements by sensors 108 of FIG. 2 include pK values, pH values, threshold values, context measures and soft indicators.

In the preferred embodiment, after performing repeated reference runs or experiments per above explanation, reference models 140 are learned by learning module 120 by processing time series data of reference biomarker measurements 110A-Z. These measurements are collected at times t₁, t₂, . . . , tᵢ from reference in vitro samples 103A-Z. More specifically, processing module 120 learns a reference model 140A1 that is able to predict a labeled set of mitochondrial dysfunction or dysfunctions, such as “D1” in Table 6 above, present in in vitro samples 103A-Z.

The set of mitochondrial dysfunction/dysfunctions are deliberately induced into in vitro cultures 102A-Z as a result of the introduction of the specific mitochondrial inhibitor, inducer, stress/stressor or insult that is known for causing the respective mitochondrial dysfunction/dysfunctions. Recall from above, that the mitochondrial inhibitor is preferably introduced in varying dosages. Model 140A1 may be learned or trained using a specific supervised machine learning algorithm such as linear regression, logistic regression, support vector machines (SVM), decision trees, random forests, neural networks and the like. Trained model 140A1 is able to predict whether the labeled mitochondrial dysfunction, for example D1, that the model was learned/trained to predict, is present or not in an unseen biomarker measurement processed in the future.

In practice, a number of additional reference models 140A2, 140A3, . . . may be learned each predicting the same labeled set D1 of mitochondrial dysfunction/dysfunctions, and each using a different machine learning algorithm. For example, reference model 140A1 may use linear regression to predict D1, model 140A2 may use logistic regressions to predict D1, model 140A3 may use Support Vector Machines (SVM) to predict D1, model 140A4 may use random forests to predict D1 and so on.

Then, learning module 120 may use the output of this ensemble of models to make a determination as to whether a biological sample from which a biomarker measurement is obtained, has mitochondrial dysfunction/dysfunctions D1 or not. For this purpose, learner 120 may choose the prediction that is most “agreed upon” by the ensemble of models 140A1, 140A2, 104A3, 140A4 above to predict D1. Specifically, if the majority of the models predict that the sample contains D1, then the sample is presumed to contain mitochondrial dysfunction(s) D1, otherwise not.

Similarly, by performing another set of runs or experiments with another mitochondrial inhibitor, processing module 120 deploys the above or another combination of machine learning algorithms to learn reference models, for example, models 140A5-A10 that are able to predict another labeled set “D2” of mitochondrial dysfunction/dysfunctions in in vitro cultures 102A-Z. Again, D2 is present in the cultures because of the introduction of the respective mitochondrial inhibitor that is known for causing dysfunction(s) D2. Recall from above, that the mitochondrial inhibitor is preferably introduced in varying dosages.

Trained reference models 140A5-A10, each using a different machine learning algorithm, can predict the presence/absence of D2 in a given biological sample. As before, learner 120 determines the sample to possess D2 if the majority from the ensemble of models 140A5-A10 agree on the presence of D2, otherwise not. Alternatively, learner 120 may use some other suitable metric for voting on the models other than the majority. For example, prediction from certain models in the ensemble may be preferred over others by using weights. As such, an overall weighted and normalized probability from the ensemble for predicting a dysfunction D2 may be produced using the following equation:

$$Eq . 1$$

$${\sum }_{1}^{No . of models predicting D 2} \frac{Weight of model predicting D 2 * predicted prob . of D 2}{No . of models predicting D 2}$$

In a similar fashion, as many reference models 140A11-1N are learned as desired to predict respective labeled mitochondrial dysfunction/dysfunctions in biological samples.

In some cases, times t₁, t₂, . . . , tᵢ at which reference biomarker measurements are taken are selected to mark distinct stages, transitions, reaction periods or still other important times in the bioprocess of interest that the reference in vitro culture is undergoing. These include the lag, growth, stationary and death phases of the cell-culture. FIG. 3 shows the typical lag, growth or exponential, stationary and death phases of a cell-culture. Times t₁, t₂, . . . , tᵢ at which the instant reference samples are drawn for measurements may thus be taken to coincide with the beginning and/or end of one or more stages. Alternatively, or in addition, they may coincide with any number of time instants during the one or more stages shown in FIG. 3 of the cell-culture. As already noted, typically the mitochondrial inhibitor is introduced at the start of the stationary phase of the cell-culture. As such, times t₁, t₂, . . . , tᵢ at which the instant reference samples are drawn for measurements will be times since the start of the stationary phase of the culture.

The biomarker measurements may be made on short time scales in comparison to Gene-Protein-Reaction (GPR) times although this is not a requirement. Hence in advantageous embodiments times t₁, t₂, . . . , tᵢ at which reference biomarker measurements are taken at a frequency of at least once every month, at least once every two weeks, at least once every 10 days, at least once every 5 days, at least once every 2 days, at least once every day, at least once every 12 hours, at least once every hour, at least once every 30 minutes, at least once every 10 minutes, at least once every 5 minutes, at least once every minute, at least once every 30 seconds, at least once every 10 seconds, at least once every 5 seconds, at least once every second, at least twice every second, at least 5 times every second, at least 10 times every second, at least 20 times every second, at least 50 times every second, at least 100 times every second, or more.

As already mentioned, diagnostic platform 100 of FIG. 2 can employ one or more learning methods. Some particularly useful methods in the embodiments of the present invention include Artificial Intelligence (AI) methods, Hidden Markov methods and Deep Learning (multi-layered neural network) methods.

Let us now look at the machine learning process employed by system 100 in greater detail. Specifically, the learning process is embodied by learning phase 52 of protocol 50 of FIG. 1 discussed above. The below explanation is provided rigorously for some of the machine learning techniques. Based on the framework provided below, one skilled in the art will be able conceive additional techniques of machine learning to practice the instant teachings.

### Learning

Recall that processing module 120 learns a reference model, for example reference model 140A1 of FIG. 2, by deploying one or more machine learning algorithms. The reference model is capable of predicting a given labeled set of mitochondrial dysfunction/dysfunctions such as D1 induced in reference in vitro cultures 102 as a result of the varying-dosage based introduction of a specific mitochondrial inhibitor.

One way to state the objective of the learning process is for reference model 140A1 to extract the most “conserved” or representative biomarker fingerprint or signature across the most number of reference in vitro samples 103 of different cell-cultures 102 from different cell-lines. If such a fingerprint is also not present in the uninhibited reference in vitro culture and its predicted value is above a predetermined threshold, then it is indicative of the presence of the corresponding labeled mitochondrial dysfunction(s) D1. Stated differently, reference model 140A1 is trained to make a prediction of the presence of dysfunction D1 based on the most conserved/representative biomarker fingerprint/signature or pattern across a variety biomarker measurements 110A-Z made on samples 103A-Z drawn from a variety of cultures 102A-Z at a variety of times tᵢ under a variety of conditions such as varying dosages di of the mitochondrial inhibitor used.

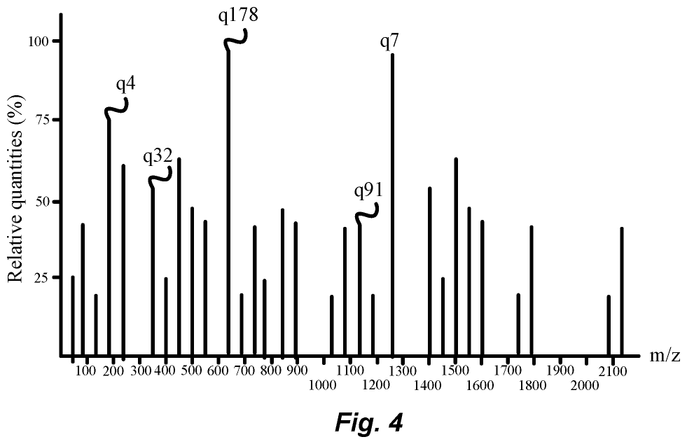

The reference biomarker measurements consist of the quantities of analytes measured, examples of which were given in Tables 3-5 above, and performed by a measuring instrument(s) 108 such as a high-throughput mass spectrometer. FIG. 4 provides an exemplary read-out or mass spectrum of measurements of some of the analytes whose measured quantities we will only denote by q1, q2, . . . instead of using their full medical names as provided in Tables 3-5. This is to avoid detraction from the main principles being taught. X-axis of FIG. 4 represents the familiar mass-to-charge ratio (m/z) and y-axis represents the relative quantities of the analytes. The measurements of some of the analytes are marked in FIG. 4 as shown. A readout from a sample, such as that shown in FIG. 4 constitutes a biomarker measurement 110 of FIG. 2 according to the present teachings.

Linear Regression:

As stated, one of the algorithms that may be used to train or learn reference model, such as model 140A1 of FIG. 2, is linear regression with supervised learning. Those skilled in the art will appreciate that linear regression is given by the equation:

Y=f(X)+ε Eq. 2

Here X specifies the input or independent variables, Y is the output or dependent variables or responses, f describes the relationship between X and Y and e is the random error term (positive or negative) with a mean of zero.

According to the present technology, each biomarker measurement 110 such as that shown in FIG. 4, constitutes an observation x∈X. X is a multi-dimensional matrix or tensor of the form [(t1, t2, . . . ti), (d1, d2, . . . dj), (c1, c2, . . . cl)], where each entry of X is a vector of the form [q1, q2, . . . qk]. Here, t1, t2, . . . ti are the times since exposure of cultures 102A-Z to a specific mitochondrial inhibitor or perturbation at which respective samples 103A-Z were drawn per above discussion. d1, d2, . . . dj are the dosage amounts in which the mitochondrial inhibitor was introduced and q1, q2, . . . qk are the quantities of specific analytes that were measured from samples 103A-Z and examples of which were given in Tables 3-5 (see also FIG. 4 and related explanation).

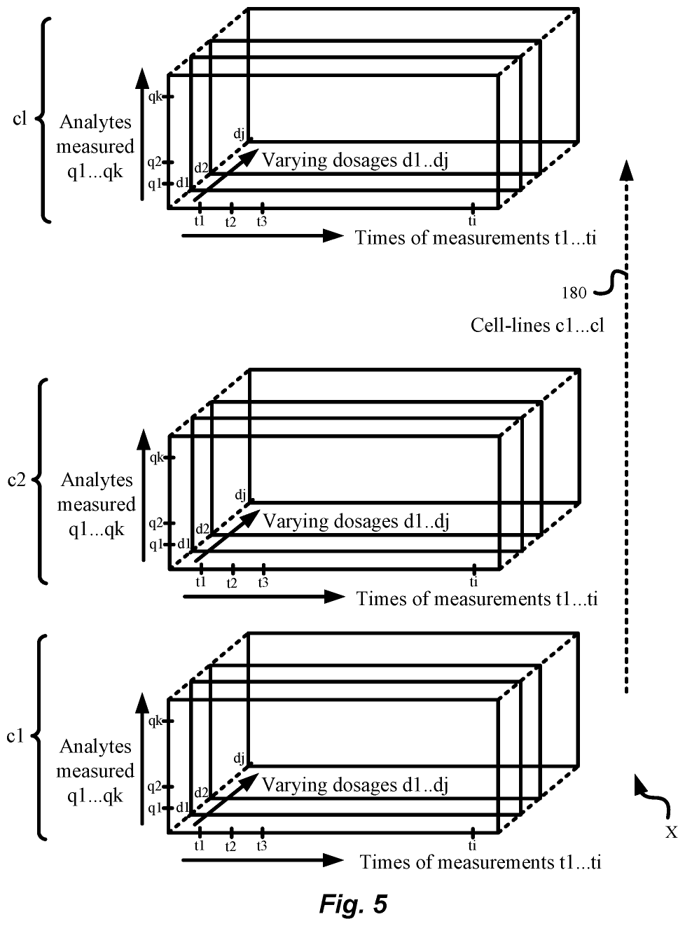

Further, variable c_{1 . . . l} (where l is the small-case alphabetical letter “l” as in “l”ima) is used to denote the number of cell-lines from which cultures 102A-Z for a given set of runs or experiments are grown. Recall that the cultures are grown from different cell-lines and are exposed to different dosages of the inhibitor/stressor. Then samples are taken from the cultures at different times and biomarker measurements 110 are taken from those samples. The resulting 4^{th} order tensor X is visualized in FIG. 5 in a pictorial form by “piling up” three-dimensional hyperrectangles or parallelepipeds composed of rectangles as shown. The reader is cautioned not to judge the pictorial illustration of FIG. 5 which is provide for explanatory purposes too strictly. This is because the 4^{th} order tensor X is inherently multi-dimensional and is hard to illustrate in the two-dimensional drawing of FIG. 5. As such, FIG. 5 should be taken as an intuitively convenient rather than a mathematically strict representation of tensor X.

The hyperrectangles shown in FIG. 5 are built from piling up two-dimensional matrices shown as rectangles whose rows indicate times of measurements t1-i and whose columns indicate the relative quantities q1-k of individual analytes being measured as shown in the exemplary illustration of FIG. 4. Note that each column of the two-dimensional matrices constitutes a biomarker measurement 110A-Z of the present teachings and is a vector x of measured relative quantities of the form [q1, q2, . . . qk] introduced above and shown in FIG. 4. Also, from FIG. 4 we know that these quantities are relative quantities of the analytes from the overall sample as measured by a preferably high-throughput and high-resolution mass spectrometer.

Each matrix or rectangle in FIG. 5 is populated for a given dosage quantity from dosages d1-j of the mitochondrial inhibitor being used and while using a given cell-line from cell-lines c1-l from which the in vitro cultures were grown for the set of experiments. Piling up individual matrices or rectangles gives us a three-dimensional rectangle or a hyperrectangle or a parallelepiped. Thus, each hyperrectangle is populated with vectors [q1, q2, . . . , qk] for varying times t1-i, for varying dosages d1-j but for a given cell-line from cell-lines c1-l from whom the in vitro cultures used in the set of experiments were grown. Finally, piling up individual hyperrectangles as shown by the upward facing vertical dotted arrow 180 gives us the multi-dimensional data structure constructed for our 4^{th} order input tensor X. Thus, tensor X has i*j*l entries of vectors x each of the form [q1, q2, . . . qk] and thus having k entries each.

Based on this framework of multi-dimensional data, one skilled in the art will be able to conceive extensions of the design to incorporate additional dimensions. More specifically, one can conceive extending our tensor X of FIG. 5 with additional dimensions that represent additional varying conditions of the experiments/runs. For example, one can have another dimension tempi to represent varying temperatures that cultures 102 were subjected to at which samples 103 were drawn to make measurements 110. Similarly, one can have a dimension pi to represent the pressure that the samples were subjected to at which the measurements were taken, another dimension gi to represent exposure to a gas specie, etc. However, to not detract from the key principles being taught, the below teachings will be based on the 4^{th} order tensor X shown in FIG. 5 and discussed above.

Y in Eq. (2) above is the labeled mitochondrial dysfunction or the labeled set of mitochondrial dysfunctions being predicted by learning system 100 of FIG. 2. System 100 is trained on “labeled” data constructed for tensor X as shown in FIG. 5. Examples of mitochondrial dysfunctions and their associated labels Dm were given in Table 2 above. These dysfunctions are induced as a result of the introduction as perturbations or stresses of respective mitochondrial inhibitors specified in the column named “Mitochondrial dysfunction induced” of Table 2 above.

Though there may be more than one dysfunctions induced by a given inhibitor as shown in Table 2 above (for example D5), we will collectively refer to all the dysfunctions associated with a given mitochondrial inhibitor by a singular label Dm and at times refer to these dysfunctions only in the singular, as dysfunction. It is these labeled dysfunction(s), such as D1, that model 140A1 predicts as dependent variables or responses Y in Eq. (2) above.

Since the present embodiment uses linear regression, Y is assumed to be linearly related to X. Further, since X has more than one values or features, the regression technique is termed as multiple linear regression, and for our tensor X above, it is given by:

Y=β₀+β₁x_{(t1,d1,c1)}+β₂x_{(t2,d1,c2)}+β_{i*j*l}x_{(ti,dj,cl)} Eq. 3A

Here, x(t_{1 . . . i}, d_{1 . . . j}, c_{1 . . . l})∈X are the input variables or features illustrated in FIG. 5 and are each vectors of the form [q1, q2, . . . qk] noted above. In the present embodiment, these are the measured quantities of the various analytes q1-k (see Table 3-5 above) constituting a biomarker measurement taken from a sample drawn at a given time ti from an in vitro culture grown from a cell-line cl (see Table 1 above) exposed to a given dosage dj of a specific/chosen mitochondrial inhibitor (see Table 2 above). Each measurement taken is a collection of read-outs of the various quantities of analytes qk such as the ones exemplarily given in Table 3-5 above and illustrated in the exemplary mass spectrum or readout of FIG. 4.

In Eq. (3A), β₀, β₁, . . . , β_{i*j*l} are the coefficients of the linear regression to be determined as further explained below. An error term ε_{1 . . . i*j*l}, is also added to Eq. (2) above and it indicates the quality of the prediction of Y by the model as compared to the actual value of Y as measured. This error term can be estimated as root-mean-squared error (RMSE) between the predicted/hypothesized and actual values of Y.

In order to train a given reference model, we shall first divide the known dataset represented by tensor X of the above discussion, into two parts. The first part preferably constitutes 80% of the total entries in X and is referred to as training data/dataset.

The second part preferably constitutes the remaining 20% entries in the dataset and is referred to as test data/dataset. In this manner, reference models 140A1-AN are trained or learned on the training dataset and are then tested on the test dataset to determine their accuracy and bias. As will be further discussed, this allows one to tune the hyperparameters of the models and address their “bias-variance tradeoff” with techniques known in the art.

Note that since Y in Eq. (3A) represents the response or predicted value of a single labeled mitochondrial dysfunction(s) for a given set of experiments, it is as such a vector of probabilities. Reference models 140A1-AN are each trained by providing it a value from D1, D2, . . . , Dm after the measurement of each biomarker measurement of relative quantities represented by vector x=[q1, q2, . . . qk]. In other words, in the exemplary case of training our model 140A1 to predict D1, one would provide model 140A1 the known label D1 in response to each observation x=[q1, q2, . . . qk]∈X corresponding to each of i*j*l vector entries x of tensor X. As such, vector Y also has i*j*l entries and in its expanded form as denoted by:

y∈Y[y_{x(t1,d1,c1)}, . . . ,y_{x(ti,d1,c1)},y_{x(t1,d2,c1)}, . . . ,y_{x(ti,d2,c1)}, . . . ,y_{x(t1,dj,c1)}, . . . ,y_{x(t2,dj,c1)}, . . . ,y_{x(ti,dj,c1)}, . . . ,y_{x(t1,dj,c2)}, . . . ,y_{x(ti,dj,cl)}],

or more simply by Y_{t1 . . . i,d1 . . . j,c1 . . . l} or still as a vector Y of the form [y1, y2, . . . , y_{i*j*l}]. Thus, a partial excerpt from an exemplary training data derived from input tensor X for system 100 may be given by Table 7 below:

TABLE 7
Training dataset for dysfunction D1 with inhibitor A8674

| No. | Y | X (tᵢ) | | X (dⱼ) | x (cₗ) | X (qₖ) |
|---|---|---|---|---|---|---|
| 1 | 0% | 1 | minute | 0 | Y79 | [symbol] |
| 2 | 0% | 5 | minutes | 0 | Y79 | [symbol] |
| 3 | 0% | 30 | minutes | 0 | Y79 | [symbol] |
| 4 | 0% | 5 | hours | 0 | Y79 | [symbol] |
| 5 | 0% | 1 | day | 0 | Y79 | [symbol] |
| 6 | 0% | 1 | minute | 1 uL | Y79 | [symbol] |
| 7 | 50% or 0.5 | 5 | minutes | 1 uL | Y79 | [symbol] |
| 8 | 100% or 1 | 30 | minutes | 1 uL | Y79 | [symbol] |
| 9 | 100% or 1 | 1 | hours | 1 uL | Y79 | [symbol] |
| 10 | 100% or 1 | 5 | hours | 1 uL | Y79 | [symbol] |
| 11 | 100% or 1 | 10 | hours | 1 uL | Y79 | [symbol] |
| 12 | 100% or 1 | 2 | days | 1 uL | Y79 | [symbol] |
| 13 | 50% or 0.5 | 1 | minute | 5 uL | Y79 | [symbol] |
| 14 | 100% or 1 | 5 | minutes | 5 uL | Y79 | [symbol] |
| 15 | 100% or 1 | 30 | minutes | 5 uL | Y79 | [symbol] |

| | | | | | |
|---|---|---|---|---|---|
| . . . | . . . | . . . | . . . | . . . | . . . |

In the above training dataset, each entry of column Y is a value in the vector Y discussed above. Each entry \hat{q} of column X(qk) with a hat “\hat{ }” denotes a vector and is an entry in input tensor X, and is a vector of measured quantities [q1, q2, . . . , qk] of the analytes in the biomarker measurement such as those shown in Tables 3-5. In other words, the training dataset consists of measured vectors \hat{q} at indices i,j,l of tensor X per above explanation. Human Caucasian retinoblastoma (Y79) from Table 1 above is used as the cell-line from which in vitro cultures are grown. Column X(d) indicates dosage of the introduction of the mitochondrial inhibitor, for example, A8674 (Antimycin A from Streptomyces sp.) from Table 2 above and Column X(t) indicates the times at which the samples were drawn for measurements.

Note that rows 1-6 correspond to the scenario when no inhibitor was exposed (zero/O state), and unsurprisingly column Y indicates a 0 or a 0% probability that dysfunction D1 is present in the corresponding culture. Row 7 indicates that at 1 minute exposure of a dosage of 1 uL of A8674, the culture is known not to have developed dysfunction D1 or still has a 0% chance, at 5 minute exposure it has a 50% chance and at 30 minutes or above, it has a 100% chance of developing dysfunction D1. However, with a dosage of 5 uL of A8674, the culture has a 50% chance of D1, at 5 minutes or above, it has a 100% of chance of having D1. Recall that it is important to have rows 1-6 in the dataset with no introduction of the inhibitor, so model 140A1 can learn what patterns or biomarker fingerprints do not correspond to D1.

Now let us understand how diagnostic platform determines the values of weights or coefficients β₀, β₁, . . . , β_{i*j*l} in Eq. (3A) above. Determining these coefficients or internal parameters is necessary in order to compute using Eq. (3A) the value of Y indicative of the presence or predictive of a specific labeled dysfunction(s) D1-m per above explanation. This allows for the training or learning of model 140A1 of the present example on a given training dataset/data. Once model 140A1 has been trained, or alternatively stated, the values of coefficients/weights β₀, β₁, . . . , β_{i*j*l} have stabilized or converged as will be taught below, Eq. (3A) is then used to predict the presence of D1 on unseen biological samples in the future.

Depending on the values of these coefficients/weights β₀, β₁, . . . , β_{i*j*l}, model 140A1 is able to estimate which observations x=[q1, q2, . . . qk]∈X corresponding to each of i*j*l vector entries x of tensor X are more important than others. Because of varying scales/units of features/observation x, it is generally ill-advised to ascribe the importance of an observation x on the basis of the value of its estimated coefficient βₓ∈{β₀, β₁, . . . , β_{i*j*l}}computed using Eq. (3A) above. However, one can use standardized regression coefficients for comparison.

Those skilled in the art will appreciate that standardized regression coefficients are the estimates resulting from a regression analysis that have been standardized so that the variances of dependent and independent variables are 1. More rigorously, they mean how many standard deviations a dependent variable will change, per standard deviation increase in the predictor variable. A standardized coefficient β* is derived from estimated coefficient βₓ by using the formula:

β*ₓ=βₓ·σₓ/σ_{y} Eq. 3B

In other words, multiply the estimated coefficient from Eq. (3A) by the standard deviation of the predictor variable x∈X and divide it by the standard deviation of the outcome variable y∈Y to arrive at the standardized coefficient. Then, values of standardized coefficients β* that are above a predetermined threshold signify the biomarker fingerprint/signature that is indicative of mitochondrial dysfunction D1.

Furthermore, these selected standardized coefficients β* may then be used to reduce/select the observations and consequently those analytes that are more predictive of dysfunction D1 than others. This feature selection/reduction allows one to choose more specialized or targeted sensing equipment during targeting phase 54 of protocol 50 (see FIG. 1). Also, this leads to economies of scale or practice by not tying up expensive equipment, such as a high throughput and high-resolution spectrometer, by requiring it to only measure those analyte peaks that matter most for prediction of specific mitochondrial dysfunctions.

Stated differently, as learning module/engine 120 trains models to predict labeled mitochondrial dysfunctions, uncorrelated or noisy measurements are dropped out. The models can thus detect patterns of analytes or fingerprints that best predict the labeled dysfunctions based on the most relevant training data obtained during learning phase 52 of FIG. 1 in vitro. These models can then be applied to a reduced set of measurements that may be available in vivo during targeting phase 54 and without compromising the predicting prowess of the models.

Ordinary Least Squares:

In one embodiment, system 100 learns reference models such as model 140A1 by employing ordinary least squares (OLS) for solving Eq. (3A) above. This approach can lead to a completely analytical and closed-form solution. More rigorously, Eq. (3A) is written with a residuals term e for some estimate of model parameters 3 as follow:

Y=β₀+β₁x_{(t1,d1,c1)}+β₂x_{(t2,d1,c2)}+β_{i*j*l}x_{(ti,dj,cl)}+e_{i*j*l}⇒e_{i*j*l}=Y−(β₀+β₁x_{(t1,d1,c1)}+β₂x_{(t2,d1,c2)}+β_{i*j*l}x_{(ti,dj,cl)}) Eq. 4

Or more simply, by dropping the subscripts and including a hat “\hat{ }” over coefficients/parameters vector 3 to indicate a vector, we get:

e=Y−X\hat{β} Eq. 5

The above assumes that the first value x(0,0,0) in tensor X is a unit vector of the form [1,0,0 . . . ]. Such an assumption leads to β₀ being the intercept of the regression in the hyperspace of our tensor X. It follows from Eq. (5) above that:

$$\begin{matrix}{e}^{T} e = {( Y - X \hat{β} )}^{T} ( Y - X \hat{β} ) \\ = {Y}^{T} Y - {Y}^{T} ( X \hat{β} ) - {( X \hat{ β} )}^{T} Y + {( X \hat{β} )}^{T} ( X \hat{β} ) \\ = {Y}^{T} Y - {( X \hat{β} )}^{T} Y - {( X \hat{β} )}^{T} Y + {( X \hat{β} )}^{T} ( X \hat{β} ) \\ = {Y}^{T} Y - 2 {( X \hat{β} )}^{T} Y + {( X \hat{β} )}^{T} ( X \hat{β} ) \\ = {Y}^{T} Y - 2 \hat{β} {X}^{T} Y + {\hat{β}}^{T} {X}^{T} X \hat{ β}\end{matrix}$$

Here superscript “T” represents a transpose operation and for our tensor X, it is obtained by preserving the first index “i” and by swapping subsequent indices “j” and “l” while also preserving each vector entry x∈X in its original form. In other words, each vector x∈X is preserved as is, the vectors at indices “i” are also retained as they are, and vectors at each value of indices “j” and “l” are swapped. For a tutorial on higher order tensor operations, the reader is referred to “Higher Order Tensor Operations And Their Applications” by Emily Miller of The College of New Jersey and Scott Ladenheim of Syracuse University, published in TCNJ Journal Of Student Scholarship Volume XI, dated 2009.

To determine the coefficients \hat{β}, we minimize the sum of squared residuals with respect to the parameters:

$$\begin{matrix}\frac{\partial [ {e}^{T} e ]}{\partial β} = 0 \\ = - 2 {X}^{T} Y + 2 {X}^{T} X \hat{β}\end{matrix}$$

Utilizing the identity

$$\frac{\partial {a}^{T} b}{\partial a} = b$$

for vectors a and b, we get:

X^{T}Y=X^{T}X\hat{β} Eq. 6

The above directly leads to:

\hat{β}=(X^{T}X)⁻¹X^{T}Y, Eq. 7

provided (X^{T}X) is invertible or non-singular. Eq. (6) and (7) above are in the form of “Normal Equations” for coefficients \hat{β} and can be solved analytically since the values of X and Y are known. Thus, in the present embodiment, model 140A1 is trained by using tensor X of measurements/observations of biomarker measurements in Eq. (7) above to compute the values of coefficients or parameters or weights f of the linear regression.

Then, given a new and unseen value of tensor X containing test data, model 140A1 is able to predict the value of a mitochondrial dysfunction/dysfunctions D1 (Table 2 above) by using Eq. (3A) above. Recall that we had partitioned input tensor X into a training dataset and a test dataset. Thus, we can train model 140A1 on the training dataset and compute its bias and accuracy on the test dataset and thusly tune its hyperparameters, as will be further discussed below.

Since the training data contains values of Y that are normalized as probabilities between 0 and 1 (see Table 7 above), Eq. (3A) will produce values of Y that are also normalized between 0 and 1. As such, these values are interpreted to mean the probability of the presence or absence of dysfunction D1 in the test data. Thus, in the present embodiment, we are using linear regression for classification.

In other words, if a given value y∈Y=[y1, y2, . . . y_{i*j*l}] is 0.7 then there is a 70% chance that the corresponding measurement in input dataset X is indicative of dysfunction D1 in corresponding cell-culture from which measurement sample was obtained. Recall that input tensor X contains i*j*l entries of vectors x, each of the form [q1, q2, . . . , qk]. Furthermore, the test dataset derived from tensor X may contain one or more vectors x=[q1, q2, . . . qk]∈X each producing a corresponding value y∈Y. Although as noted above, it is advisable to partition input tensor X such that test dataset is about 20% of the size of the overall input data X.

Gradient Descent:

If the number of features or in our case vectors in input tensor X is very large, for example, greater than 10,000, then Eq. (7) can have a high computation time. Therefore, in another embodiment, the technique of gradient descent is employed to compute coefficients \hat{β}.

In this embodiment, a cost function is iteratively minimized to arrive at the estimated values of coefficients β₀, β₁, . . . , β_{i*j*l}. More rigorously, let us denote by H_{β} the predicted or hypothesized value of a given dysfunction, for example dysfunction D2 (Table 2 above), by a reference model, such as model 140A5. The purpose of doing this is so that we can differentiate the predicted or hypothesized value h_{β}∈H_{β} based on coefficients \hat{β} from the corresponding actual value y∈Y as measured or known in the dataset.

In other words, we will denote the left-hand side of Eq. (3A) by H_{β} and rewrite it as:

H_{β}=X\hat{β} Eq. 8

It should be apparent that each of vectors H_{β} and \hat{β} have i*j*l entries. In other words, h_{β1 . . . i*j*l}∈H and y_{1 . . . i*j*l}∈Y. We will now define the cost or loss function J(β) that associates a cumulative cost to the difference between the predicted value h_{β1 . . . i*j*l}∈H obtained by Eq. (8) and the corresponding actual value y_{1 . . . *j*l}∈Y as known. Specifically,

$$J ( β ) = \frac{1}{i * j * l} {\sum }_{a = 1}^{i * j * l} {( {h}_{β} {x}_{a} - {y}_{a} )}^{2} \qquad Eq . 9$$

By minimizing J(β) in successive iterations, we can obtain estimates of coefficients β₀, β₁, . . . , β_{i*j*l} to be used in Eq. (3A) above on test or unseen data in the future. Specifically, we perform gradient descent to find the local minimum of J(β) using the equation:

$${β}_{1 … i * j * l} = {β}_{1 … i * j * l} - α \frac{\partial }{\partial {β}_{1 … i * j * l}} J ( β ) \qquad Eq . 10$$

Here α is the learning rate hyperparameter indicative of how aggressively the process tries to find the minimum or “convergence”. It can be chosen very conservatively at the outset and may be gradually increased later to achieve convergence quicker. An exemplary initial value of a may be taken as 0.000001. Care should be taken not to increase a too sharply or the algorithm may overshoot the minimum and diverge.

According to the present design, the gradient descent process of estimating values of coefficients β₀, β₁, . . . , β_{i*j*l} is given by the following pseudocode:
Step 1: Initialize all coefficients β₀, β₁, . . . , β_{i*j*l} to an initial value, for example 0.
Step 2: Compute predictions H_{β} by Eq. (8)
Step 3: Compute loss J(β) by Eq. (9)
Step 4: Update coefficients β₀, β₁, . . . , β_{i*j*l} by Eq. (10)
Step 5: Are the values of coefficients β₀, β₁, . . . , β_{i*j*l} similar to their values from the previous iteration within a predetermined threshold, for example, 0.1 ? If Yes, Exit. If Not, go back to Step 2.

Continuing with our above example, once the values of coefficients β₀, β₁, . . . , β_{i*j*l} are stabilized or in other words gradient descent has converged, then our model 140A1 is said to have been trained to predict dysfunction D1, using Eq. (8) above. To address the familiar problem of “over-fitting”, a regularization term may be added to cost function J(β) of Eq. (9) as follows:

$$J ( β ) = \frac{1}{2 * i * j * l} [ {\sum }_{a = 1}^{i * j * l} {( {h}_{β} {x}_{a} - {y}_{a} )}^{2} + λ {\sum }_{b = 1}^{i * j * l} {β}_{b}^{2} ] \qquad Eq . 11$$

Here λ is the regularization coefficient and is another hyperparameter that can be tuned. A higher X will more harshly penalize large coefficients that could lead to potential overfitting.

In alternative embodiments, Newton's method may be utilized to minimize loss J(β) and estimate the values coefficients β₀, β₁, . . . , β_{i*j*l}. Using the above-provided framework and our input tensor X, one skilled in the art is also able to apply other variations of these algorithms and related machine learning techniques, such as batch gradient descent, and the like. Furthermore, and as already noted, one can extend input tensor X to include additional dimensions that represent environmental conditions, or other variables of the experiments.

Logistic Regression:

In still other embodiments, the machine learning technique of logistic regression may be employed as a natural choice for classification of samples into those that have the labeled dysfunction and those that do not. Again, since input X has multiple features, the technique may be termed as multiple logistic regression. To use logistic regression, we define p=E(y∈Y|x∈X) as the conditional probability y for some value of regressors x∈X. In other words, p defines the probability that a dysfunction Dm is present in a given biomarker measurement x measured from a sample. Then, logistic or logit regression given our tensor X above is expressed by the equation:

$$ln [ \frac{p}{1 - p} ] = {β}_{0} + {β}_{1} {x}_{( t 1 , d 1 , c 1 )} + {β}_{2} {x}_{( t 2 , d 1 , c 2 )} + … + {β}_{i * j * l} {x}_{( ti , dj , cl )} \qquad Eq . 12$$

Once again, gradient descent is used to compute the values of coefficients or weights β₀, β₁, . . . , β_{i*j*l}. More specifically, denoting hypothesized values again by h_{β}∈H_{β}, we can rewrite Eq. (12) above as follows:

$$ln [ \frac{{h}_{β}}{1 - {h}_{β}} ] = {β}_{0} + {β}_{1} {x}_{( t 1 , d 1 , c 1 )} + {β}_{2} {x}_{( t 2 , d 1 , c 2 )} + … + {β}_{i * j * l} {x}_{( ti , dj , cl )} \qquad Eq . 13$$

We can then define the regularized cost function as:

$$j ( β ) = - \frac{1}{i * j * l} {\sum }_{a = 1}^{i * j * l} [ {y}_{a} ln [ {h}_{β} {x}_{a} ] + ( 1 - {y}_{a} ) ln [ 1 - {h}_{β} {x}_{a} ] ] + \frac{λ}{2 * i * j * l} {\sum }_{b = 1}^{i * j * l} {β}_{b}^{2} \qquad Eq . 14$$

As with linear regression, here λ is the regularization coefficient and is a hyperparameter that can be tuned to control how harshly overfitting is penalized. We can then use the following pseudocode to compute the values of coefficients β₀, β₁, . . . β_{i*j*l}.
Step 1: Initialize all coefficients β₀, β₁, . . . , β_{i*j*l} to an initial value, for example 0.
Step 2: Compute predictions H_{β} by Eq. (13)
Step 3: Compute loss J(β) by Eq. (14)
Step 4: Update coefficients β₀, β₁, . . . , β_{i*j*l} by Eq. (10)
Step 5: Are the values of coefficients β₀, β₁, . . . , β_{i*j*l} similar to their values from the previous iteration within a predetermined threshold, for example, 0.1 ? If Yes, Exit. If Not, go back to Step 2.

In alternative embodiments, Newton's method may be used to minimize loss J(β) and to estimate the values coefficients β₀, β₁, . . . , β_{i*j*l}. After computing the values of β₀, β₁, . . . , β_{i*j*l}, one can now easily compute the value of p by using Eq. (12) above. One then defines a threshold value, for example 0.5 or 0.7. If the value of p is greater than the threshold value then the mitochondrial dysfunction in question, D1 from our prior example, is assumed to exist in the sample, otherwise not. In other words, the output is a labeled dysfunction Dm if p>threshold value, otherwise not. The library of reference models 140A1-AN trained according to above teachings is stored in database 170 as shown in FIG. 2.

Using the framework provided above, one skilled in the art can also apply the techniques of support vector machines (SVM) and/or decision trees to classify samples into those that have the labeled dysfunction and those that do not. With the above-provided tensor X and the related teachings of the learning framework, one can conceive the implementation details of SVM and decision trees-based learning models to practice the instant principles. As such, the SVM and decision trees implementations are not discussed in detail in this disclosure. Furthermore, one will also be able to compute the R² and RMSE metrics for the models to determine their efficacy on test data.

One is also able to apply the familiar technique of cross-validation to more effectively fine-tune the models and their hyperparameters than just by statically partitioning input tensor X as described above. In other words, input tensor X may be partitioned into N folds or parts with training done on the first N−1 folds followed by testing on the N^{th} (“held-out”) fold. This is followed by training on the 2^{nd} to N^{th} folds combined followed by testing on the 1^{st} fold, and then training on 3^{rd} to N^{th} and the 1^{st} folds combined followed by testing on the 2^{nd} fold, and so on. Cross-validation provides for a superior error-estimation, removal of bias and reduction of variance of the models.

Additionally, the techniques of deep learning may also be applied to learn models 140A1-AN of FIG. 2. In particular, Deep Neural Networks (DNN) may also be implemented to train the models. By utilizing an ensemble of DNN's with 3 or more hidden layers, models 140A1-AN are able to classify an input biomarker measurement as containing or not, a known mitochondrial dysfunction(s) Dm. Examples of dysfunctions Dm were given in Table 2 above. The reader is referred to the reference entitled, “Deep biomarkers of human aging: Application of deep neural networks to biomarker development” by Putin et al. published in AGING, Vol. 8 No. 5, dated May, 2016 for a framework for using DNN's for classification problems in aging research.

According to the present design, the biomarker fingerprint or pattern of relevant analytes in response to the inhibition of one or more mitochondrial functions by a mitochondrial inhibitor, may be conserved/represented (and observed/measured) across a range of cell-cultures. The cultures may be drawn from a diverse array of cell-lines and across varying times of the growth of the cultures. Further, the biomarker fingerprint will be absent or not conserved in uninhibited or normal cell-cultures. As explained, the determination of the most conserved reference biomarker fingerprints is performed by reference models 140A1-AN.

Since each reference model is trained to predict a specific dysfunction, ultimately the output of system 100 for a given unseen dataset consisting of one or more vectors x=[q1, q2, . . . qk] is a ranked list of dysfunctions as predicted for each vector x in the dataset. The ranking of the dysfunctions can be in the order of the strength/probability of the presence of a certain dysfunctions as predicted by the reference models trained per above explanation.

For example, if for a given unseen dataset consisting of a single vector x, the ensemble of models 140A1-A4 predicts D2 with an overall weighted/normalized probability from Eq. (1) above of 85%, and the ensemble of models 140A5-12 predict D4 with an overall weighted/normalized probability from Eq. (1) above of 75%, then system 100 will produce a ranked list of dysfunctions D2 with a probability of 85%, followed by D4 with a probability of 75% for vector x, and so on. Similarly, if the unseen data has more than one vectors x, such a ranked list can be produced for each of those vectors x.

Note that in practical clinical settings, unseen dataset derived from an in vivo subject/patient as will be explained below, will likely consist of one vector x. It makes sense that if there are more than one vectors x of unseen data, that they are all derived from the same in vivo subject/patient. In such a scenario, the vectors x may be derived from the same patient at different points in time.

The above-described techniques of learning of the present teachings make frequent use of matrix manipulations and are naturally suited for deployment on Graphical Processing Unit (GPU) based architectures. These include GPU architectures available from vendors such as Nvidia, Advanced Micro Devices (AMD), ARM Holdings, Broadcomm, Intel, Qualcomm, etc., as well as cloud-based GPU virtual services such as Google Cloud Platform, Amazon Web Services, IBM Cloud and the like. Moreover, the algorithms and the mathematical framework provided above may be implemented in a number of programming environments of choice.

These include TensorFlow, Caffe, Matlab, R, Azure, Apache Singa, H20, Scikit-Learn, etc. as well as general purpose programming languages including C, C++, Java, Python, etc.

### Targeting

Having described above in detail the training or learning of models 140A1-AN to predict specific mitochondrial dysfunctions Dm, let us continue our discussion of testing with in vivo samples and refer back to FIG. 2. This testing with in vivo samples is referred to as targeting in the present design and is designated by targeting phase 54 of protocol 50 of FIG. 1 presented earlier.

During targeting, models 140A1-AN that were trained on in vitro data obtained from in vitro samples 103A1-AZ are used to make predictions on target in vivo samples. Of course, the dysfunctions thus predicted by the models are the same as the ones that the models were trained on. The in vivo target samples are obtained from target patients that are known to have genetic mitochondrial diseases characterized by various mitochondrial dysfunctions. These diseases are also referred to as simply mitochondrial diseases for short. In addition, or alternatively, the patients may be known to have these genetic mitochondrial diseases diagnosed from their sequenced genomic data.

To explain targeting further, FIG. 2 also shows target/targeting in vivo subjects/patients or biological entities 152A, 152B, . . . , 152X from which respective in vivo samples 153A, 153B, . . . , 153X are drawn. Types of in vivo samples that may be obtained from target subjects/patients include blood or blood components, urine, stool samples, pleural fluid, ascites, sputum, tissue, plasma, tears, sweat, saliva, etc. As would typically be the case, only one sample 153A is being shown drawn from subject 152A, only one sample 153B from subject 152B and so on, although that is not a requirement. Analogously to the in vitro samples of the learning phase, each sample 153A-X is measured by a respective sensor or measuring instrument 158A-X that is preferably a high-throughput high-resolution mass spectrometer. Further, sensors 158A-X may be common/shared for all the in vivo samples and/or in vivo and in vitro samples as shown by dotted line 109.

Target biological entities or subjects 152A-X from whom respective in vivo samples 153A-X are drawn, are also subjected to respective target conditions 154A-X as shown. Specifically, target in vivo samples 152A-X may be live humans, plants, animals, organisms, or any other biological entities in their respective natural in vivo environments or habitats 154A-X. Targeting conditions 154 in practice are the clinical conditions in which samples 153 are drawn from the patients/subjects.

In an analogous fashion to reference measurements 110A-Z explained above, sensor systems 158A, 158B, . . . , 158X gather target biomarker measurements 160A, 160B, . . . , 160X generated from target in vivo samples 153A, 153B, . . . , 153X respectively. Each of the in vivo targets/subjects 152A-X from whom respective in vivo samples 153A-X are extracted is known to have a genetic/mitochondrial disease(s) characterized by mitochondrial dysfunction(s). Such a diagnosis for patients/subjects 152A-X may have been made on their sequenced genomic data or DNA sequencing data shown by reference numerals 156A-X respectively in FIG. 2. Sequenced genomic data or simply genomic data 156A-X may have been obtained from these subjects/patients using DNA sequencers/sequencing devices available in the art. It contains the known genetic defects that are causal of the genetic/mitochondrial diseases known to exist in these patients. However, in other cases these patients may not exhibit a genetic defect or the corresponding gene may not have expressed itself, but still the patients are known to have a mitochondrial disease based on other clinical diagnosis.

According to the instant design, target in vivo biomarker measurements 160A-X are processed during targeting by our library of reference models 140A1-AN trained above. During targeting, the reference models predict the presence of labeled dysfunctions Dm (see Table 2 above), by analyzing target biomarker measurements 160A-X originating from respective samples 153A-X of respective patients 152A-X. Let us consider that based on target biomarker fingerprints detected by models 140A1-AN in measurements 160A-X per above teachings, a dysfunction (for example D2) is predicted to be present in a statistically significant number of matching or matched target patients 152A-X (for example 500 patients). All these matched patients are known to have the same genetic mitochondrial disease (for example Complex I Deficiency).

The above knowledge provides system 100 a mapping or association 130 of dysfunction D2 to the mitochondrial disease of Complex I Deficiency along with its causal genetic defects. If D2 is implicated in Complex 1 Deficiency, then this knowledge validates our learning models in that they have accurately predicted D2 in patients that are otherwise known to have the same dysfunction/disease. This knowledge/mapping/association heretofore not existing in prior art, also exposes the correlation that exists between the causal mitochondrial inhibitor and the genetic defects/patterns observed in these patients.

In a similar manner, the targeting process is carried out for all known dysfunctions Dm against the available target population to find statistically significant number of matches to known genetic mitochondrial diseases. Thus, system 100 learns a mapping or association 130 shown in FIG. 1-2 of each mitochondrial dysfunction Dm (see Table 2 above) with the corresponding genetic mitochondrial disease and its related genetic defects/pattern. This knowledge, heretofore not available in the prior art, is very useful, because it can lead to offering new therapies for those in vivo patients as will be discussed further below.

Sometimes patients suffering from a mitochondrial disease may still not show genes expressive of their disease in their genetic data or pattern. Even in such scenarios, the above mapping is useful for associating the predicted mitochondrial dysfunction to the genetic pattern that indeed is expressed by those patients. This approach detects fingerprints of mitochondrial inhibitors that correlate to a set/cohort of gene variations. This is a useful way of learning about gene functions.

Table 8 below provides a partial excerpt of an exemplary mapping 130 that may be generated during the targeting phase. Mapping 130 is also stored in database 170 of FIG. 2 along with the library of trained reference models 140A1-AN. In addition, genomic data 156A-X of respective target patients 152A-X may also be stored in database 170 although that is not a requirement.

TABLE 8
Mapping 130

| | No. of | | |
|---|---|---|---|
| | Matched | | Cause/Genetic |
| Dysfunction | Patients | Genetic Disease | Defect |
| D12 | 876 | Complex I Deficiency | Autosomal |
| D3 | 743 | Encephalomyopathy | Autosomal Recessive |
| D10 | 1242 | Neuropathy, Ataxia, & | Mitochondrial DNA |
| | | Retinitis Pigmentosa | point mutations in |
| | | (NARP) | genes associated |
| | | | with Complex V: |
| | | | T8993G, (also |
| | | | T8993C by |
| | | | some research) |

As will be described in detail in the diagnosis phase, if an exemplary patient 152C with a known genetic mitochondrial disease is predicted by system 100 to have a dysfunction D3 and if D3 has a known rescuer (for example R1), then R1 may be provided in a targeted personalized therapy for patient 152C. This is an important innovation of the present design over the prevailing art since there is a growing number of known genetic mitochondrial diseases/disorders that can benefit from the present design.

As noted above, targeting phase 54 of FIG. 1 may be carried out only on a reduced number of features or analytes that are measured based on techniques including standardized coefficients during learning phase 52. This leads to specialized measurements and economies of scale as also noted above. Furthermore, trained models 140A1-AN may be even more refined during targeting to further reduce the number of features/analytes to be measured that are most predictive of a given mitochondrial dysfunction during diagnosis phase 56 as explained further below. To accomplish this, standardized coefficients or other techniques of feature selection/reduction may again be employed. For these reasons, learning phase 52 of protocol 50 may also be referred to as an untargeted phase that leads to more focused/refined targeted/targeting phase 54 and which leads to even more focused/refined diagnosis phase 56.

Table 9 below provides a partial list of the known genetic diseases/disorders that may be benefited this way.

TABLE 9

| No. | Mitochondrial Disease Capsules: |
|---|---|
| 1 | Alpers Disease |
| 2 | Barth Syndrome/LIC (Lethal Infantile Cardiomyopathy) |
| 3 | Beta-oxidation Defects |
| 4 | Carnitine-Acyl-Carnitine Deficiency |
| 5 | Carnitine Deficiency |
| 6 | Creatine Deficiency Syndromes |
| 7 | Co-Enzyme Q10 Deficiency |
| 8 | Complex I Deficiency |
| 9 | Complex II Deficiency |
| 10 | Complex III Deficiency |
| 11 | Complex IV Deficiency/COX Deficiency |
| 12 | Complex V Deficiency |
| 13 | CPEO |
| 14 | CPT I Deficiency |
| 15 | CPT II Deficiency |
| 16 | KSS |
| 17 | Lactic Acidosis |
| 18 | LBSL - Leukodystrohpy |
| 19 | LCAD |
| 20 | LCHAD |
| 21 | Leigh Disease or Syndrome |
| 22 | Luft Disease |
| 23 | MAD/Glutaric Aciduria Type II |
| 24 | MCAD |
| 25 | MELAS |
| 26 | MERRF |
| 27 | MIRAS |
| 28 | Mitochondrial Cytopathy |
| 29 | Mitochondrial DNA Depletion |
| 30 | Mitochondrial Encephalopathy |
| 31 | MNGIE |
| 32 | NARP |
| 33 | Pearson Syndrome |
| 34 | Pyruvate Carboxylase Deficiency |
| 35 | Pyruvate Dehydrogenase Deficiency |
| 36 | POLG2 Mutations |
| 37 | SCAD |
| 38 | SCHAD |
| 39 | VLCAD |

### Diagnosis

Once models 140A1-AN of FIG. 2 have been trained and targeted per above teachings, they are ready to be deployed in the field or clinical settings as indicated by diagnosis phase 56 of protocol 50 of FIG. 1. They can be effectively used to diagnose previously undiagnosed patients of mitochondrial dysfunctions and their association with any genetic mitochondrial diseases that the patients may be possessing. This is done by analyzing a heretofore unseen biomarker measurement obtained from their clinical sample and detecting a biomarker fingerprint predictive of a known mitochondrial dysfunction and an associated/mapped genetic mitochondrial disease per above teachings. The clinical sample may consist of blood or blood components, urine, stool samples, pleural fluid, ascites, sputum, tissue, plasma, tears, sweat, saliva, etc.

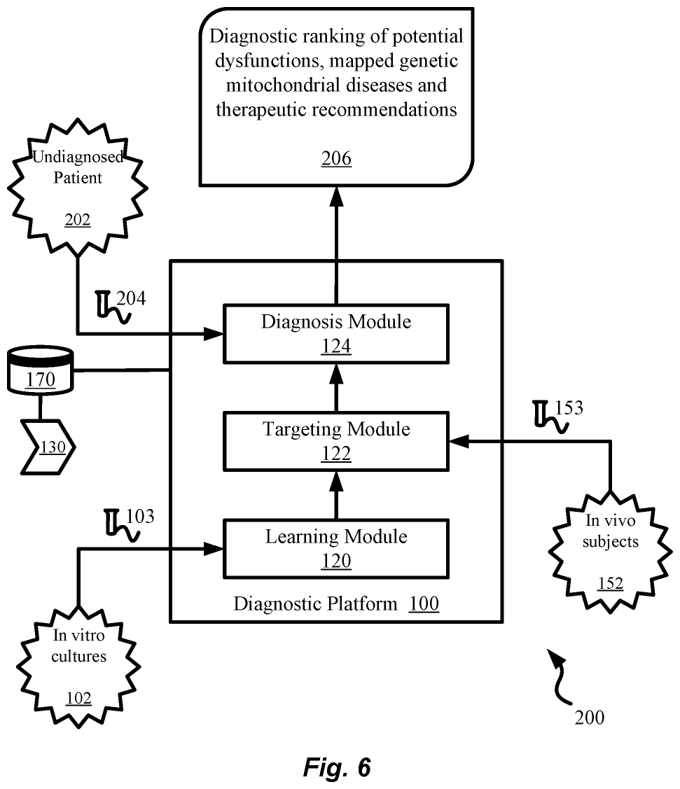

More generally, once diagnostic platform 100 processes the patient's sample, models 140A1-AN produce a list of potential mitochondrial dysfunctions in the patient and any associated mitochondrial diseases per above teachings. This diagnostic process 200 using our diagnostic platform 100 is illustrated in FIG. 6.

FIG. 6 shows further architectural details of diagnostic platform 100 of FIG. 2. In particular, platform/system 100 consists of a learning module 120 that during the learning phase learns/trains a library of trained models (not shown in FIG. 6) based on in vitro reference samples 103 obtained from in vitro cultures 103 per above teachings. Then, during targeting phase, based on target samples 153 obtained from in vivo target subjects 152, targeting module 122 develops a mapping 130 of predicted mitochondrial dysfunctions to genetic diseases known to be present in the target subjects also per above teachings.

This is followed by the diagnosis phase in which a clinical biological sample 204 is obtained from an undiagnosed patient 202. Diagnostic platform 100, specifically its diagnosis module 124 now uses the reference models trained by learning module 120 to predict a labeled mitochondrial dysfunction or dysfunctions based on an unseen biomarker measurement generated from sample 204. The biomarker measurement is made by a measuring instrument, such as a high-resolution mass spectrometer (not shown in FIG. 6). The unseen biomarker measurement contains a mass spectrum of analytes and their quantities observed in sample 204 according to above teachings.

As noted above, diagnosis phase 56 of FIG. 1 carried out largely by diagnosis module 124 of FIG. 6 may only require a reduced set of analytes to be measured from sample 204 than targeting phase 54. This is because trained models 140A1-AN may be further refined during targeting to select a smaller number of features/analytes most predictive of a given mitochondrial dysfunction than during learning phase 52.

Diagnosis module 124 uses mapping 130 of mitochondrial dysfunctions and known mitochondrial diseases to diagnose the presence of one or more mitochondrial dysfunctions/diseases in patient 202 per above discussion. Thus, a key benefit of platform 100 is that it can diagnose the presence of a potential genetic mitochondrial disease(s) in patient 202 without the requirement of DNA sequencing. In other words, if patient 202 is predicted to have D3 which is mapped to Encephalomyopathy (see Table 8 above), then platform 100 can diagnose patient 202 of Encephalomyopathy without requiring DNA sequencing. This is an important innovation over the prevailing art. Platform is also useful for patients whose sequenced genetic data is not indicative of the mitochondrial disease characterized by the mitochondrial dysfunction(s).

Furthermore, platform 100 can also be used to issue therapeutic recommendations based on any known rescuers for the mitochondrial dysfunctions associated with the diagnosed disease in the patient. Recall from learning phase above that models 140A1-AN can also be used to produce a ranked list of predicted dysfunctions Dm in a given patient, such as patient 202 of FIG. 6. As a result, platform 100, specifically its diagnosis module 124 generates a multilevel diagnostic ranking 206 for patient 202 once the diagnosis phase is complete.

Specifically, diagnostic ranking 206 consists of columns including: the stressor/insult or mitochondrial inhibitor whose introduction had induced the predicted labeled mitochondrial dysfunctions(s), the top (for example 3) ranked predicted dysfunctions, associated/mapped genetic mitochondrial disease discovered during targeting, any known causal genetic defects for the genetic disease, and any rescuer compound that is known to ameliorate the conditions associated with the corresponding labeled dysfunction(s). As shown in Table 10, diagnostic ranking 206 is indicative of the correlations that exist between the originating mitochondrial inhibitor used in the experiments and the genetic defects or genetic patterns expressed by the undiagnosed patient. This knowledge alone is helpful in suggesting improved therapies for the patient.

A partial excerpt of an exemplary diagnostic ranking 206 for our patient 202 of FIG. 6 is provided below in Table 10. Note that in some embodiments, diagnostic ranking 206 may be built in parts, with some columns populated during learning, others during targeting and/or still others during diagnosis. Further, ranking 206 may also be stored in database 170 (now shown in FIG. 6) for later retrieval and analysis. In Table 10 below, R1 is the presumed rescuer compound for dysfunction D3 (Table 2 above) while there is presumed to be no available/known rescuer compound for dysfunction D10.

TABLE 10
Diagnostic Ranking 206 for patient 202

| | | Weighted/ | | | Cause/ | |
|---|---|---|---|---|---|---|
| | Dys- | Normalized | Stressor/ | Genetic | Genetic | Known |
| Rank | function | Prediction | Inhibitor | Disease | Defect | Rescuer |
| 1 | D12 | 75% | R8875 | Complex I | Autosomal | Vitamin |
| | | | | Deficiency | | E Hydro- |
| | | | | | | quinone |
| 2 | D3 | 48% | SML1122 | Encephalo- | Autosomal | R1 |
| | | | | myopathy | Recessive | |
| 3 | D10 | 21% | O4876 | Neuropathy, | Mitochondrial | N/A |
| | | | | Ataxia, & | DNA | |
| | | | | Retinitis | point | |
| | | | | Pigmentosa | mutations in | |
| | | | | (NARP) | genes | |
| | | | | | associated | |
| | | | | | with Complex | |
| | | | | | V: T8993G, | |
| | | | | | (also T8993C | |
| | | | | | by some | |
| | | | | | research) | |

Thus, we observe from the diagnosis phase that the key benefits of the present design include:
(1) Diagnosis of a potential genetic disease without requiring DNA sequencing. This diagnosis is obtained as the top-ranking dysfunction and associated genetic disease from diagnostic ranking 206 presented above.
(2) Prognosis or potential vulnerability/risk of patient 202 to future development of genetic diseases based on the lower-ranked dysfunctions and associated genetic diseases from diagnostic ranking 206 presented above. Specifically, patient 202 may have a tendency to develop D3 and associated Encephalomyopathy, as well as D10 and associated NARP over time.
(3) A ranked list of potential therapeutic recommendations based on any known rescuer compounds that are known to alleviate the mitochondrial dysfunction(s) predicted in the patient. At least, platform 100 may be used to narrow the range of potential diagnoses or to recommend a range of diagnoses for future study.

The above are important innovations of the present design over the prevailing art.

Note that there are laboratory processes that may or may not be necessarily computer-implemented. These may include drawing reference and target samples during the learning and targeting phases respectively, as well as drawing the clinical sample during the diagnosis phase from undiagnosed patient 202 above. These may also include operating mass spectrometer(s) to obtain corresponding biomarker measurements during these phases. That is why some embodiments within the present scope will presume such tasks to be under the purview of respective computing modules i.e. learning, targeting and diagnosis modules of platform 100 as shown in FIG. 6. However, other embodiments within the present scope may practice such tasks to be manual/mechanical and outside the purview of these computing modules.

As a result, learning phase 52 of protocol 50 of FIG. 1 which contains all aspects of learning until the development of the reference models 140A1-AN is said to be “largely” implemented by learning module 120 of FIG. 1, 6. Similarly, targeting phase 54 which contains all aspects until the development of mapping 130 is largely implemented by targeting module 122 and diagnosis phase 56 is largely implemented by diagnosis module 124 of FIG. 6.

Let us now consider another practical application of the present embodiments. Mitochondrial inhibitor Rotenone (D12 in Table 2 above) is known to function by knocking out Complex 1 in mitochondria, interfering with the cells ability to consume energy. By deploying learning phase 52 (see FIG. 1) on diagnostic platform 100 of FIG. 2, let us consider that learning models 140A1, 140A2 and 140A3 are trained to predict the presence of D12 based on reference biomarker measurements of in vitro samples 102 obtained from inhibited and uninhibited cultures per above teachings.

By deploying targeting phase 54 (see FIG. 1), diagnostic platform 100 is able to find association of dysfunction D12 with the known genetic mitochondrial disease(s) involving Complex 1 deficiency (Table 9 above). Now consider that during clinical or diagnosis phase 56 (see FIG. 1), based on a blood sample a patient 202 of FIG. 6 is predicted by models 140A1-A3 to have dysfunction D12 and the associated mitochondrial disease of Complex 1 deficiency per above teachings.

Since Rotenone is implicated in causing ferroptosis due to Complex 1 deficiency (see at least reference entitled “Ferroptosis: An Iron-Dependent Form of Non-Apoptotic Cell Death”, by Dixon et al. published in Cell 149(5): 1060-1072, doi:10.1016/j.cell.2012.03.042, dated May 25, 2012) and vitamin E hydroquinone has been discovered to be a potent inhibitor of ferroptosis, this knowledge of vitamin E hydroquinone as a rescuer of D12 may be maintained by platform 100 in a rescuer table of the form <Dysfunction Dm>, <Any known Rescuer> in database 170.

Other mitochondrial dysfunctions that are linked to ferroptosis of cells may also be maintained in the rescuer table along with vitamin E hydroquinone as the rescuer. In the same rescuer table, the presumed rescuer R1 of dysfunction D3 and a null/empty field indicating no known rescuer for dysfunction D10 may also be maintained. Rescuer table may be updated by a user of platform 100 or updated via a script or still other techniques known in the art.

As a result, platform 100 and specifically its diagnosis module 124 uses the above rescuer table to populate diagnostic ranking 206 with vitamin E hydroquinone as the rescuer compound for D12 for our patient 202. Similarly, it populates R1 as the rescuer for D3, and an empty field for the rescuer for D12 in diagnostic ranking 206 for patient 202. The “Known Rescuer” column of Table 10 is then used by platform 100 and/or an associated medical professional to incorporate in personalized targeted therapies for patient 202 that were heretofore unavailable. Such a capability is tremendous contribution of the present design over prevailing art.

Since mitochondrial dysfunctions are implicated in the causes of a large number of diseases, the present techniques may be employed in the diagnosis and treatment of such diseases. These diseases include at least neurodegenerative, cardiovascular, autoimmune, neurobehavioral, psychiatric, gastrointestinal and musculoskeletal diseases. These may also include types of diabetes, metabolic syndromes, fatiguing illnesses, cancers and chronic infections.

Consequently, a non-limiting list of neurodegenerative diseases for which mitochondrial dysfunctions may be predicted by the present techniques in order to improve potential treatments include Alzheimer's disease, Parkinson's disease, Huntington's disease, amyotrophic lateral sclerosis (ALS) and Friedreich's ataxia. Similarly, a non-limiting list of cardiovascular diseases for which mitochondrial dysfunctions may be predicted by the present techniques in order to improve potential treatments include a variety of vascular conditions including atherosclerosis. A non-limiting list of autoimmune diseases diagnosable and treatable by the present techniques include sclerosis, systemic lupus erythematosus and Type 1 diabetes.

In a similar manner, a non-limiting list of neurobehavioral diseases for which mitochondrial dysfunctions may be predicted by the present techniques in order to improve potential treatments include autism spectrum disorder, schizophrenia, a bipolar disorder, a mood disorder, depression, attention deficit hyperactivity disorder (ADHD) and post-traumatic stress disorder (PTSD). A non-limiting list of fatiguing illnesses for which mitochondrial dysfunctions may be predicted by the present techniques in order to improve potential treatments include chronic fatigue syndrome and Gulf War illness. A non-limiting list of musculoskeletal diseases diagnosable and treatable by the present techniques include fibromyalgia and skeletal muscle atrophy.

The above teachings are provided as reference to those skilled in the art in order to explain the salient aspects of the invention. It will be appreciated from the above disclosure that a range of variations on the above-described examples and embodiments may be practiced by the skilled artisan without departing from the scope of the invention(s) herein described. The scope of the invention should therefore be judged by the appended claims and their equivalents.

What is claimed is:

1. A diagnostic method comprising the steps of:
(a) introducing in one or more dosages a mitochondrial inhibitor into each of one or more cell-cultures grown in vitro from one or more cell-lines, said mitochondrial inhibitor inducing a mitochondrial dysfunction into said each of one or more cell-cultures;
(b) drawing from each of said one or more cell-cultures one or more reference samples at one or more times since said introducing;
(c) making one or more reference biomarker measurements from corresponding each of said one or more reference samples;
(d) learning by a learning module one or more reference models each able to predict said mitochondrial dysfunction in an unseen biomarker measurement, said learning module comprising a microprocessor executing program instructions stored in a non-transitory storage medium coupled to said microprocessor;
(e) drawing from one or more target subjects one or more target samples in vivo and making target biomarker measurements from corresponding said one or more target samples;
(f) predicting by said one or more reference models said mitochondrial dysfunction in said one or more target subjects based on said one or more target biomarker measurements; and
(g) matching by said targeting module said mitochondrial dysfunction to a mitochondrial disease known to exist in said one or more target subjects, said matching based on a statistically significant number of subjects from said one or more target subjects who are predicted to have said mitochondrial dysfunction in (f) above.

2. The method of claim 1 utilizing said one or more reference models in an ensemble to predict said mitochondrial dysfunction.

3. The method of claim 1 utilizing at least one of multiple linear regression and multiple logistic regression in said learning in (d) above.

4. The method of claim 1 utilizing a diagnosis module for applying said one or more reference models to a clinical biomarker measurement obtained from a clinical sample of an undiagnosed patient to predict said mitochondrial dysfunction and said mitochondrial disease in said undiagnosed patient, said diagnosis module comprising a microprocessor executing program instructions stored in a non-transitory storage medium coupled to said microprocessor.

5. The method of claim 4 based on a known rescuer for said mitochondrial dysfunction, providing for a personalized targeted therapy recommendation for said undiagnosed patient.

6. The method of claim 5 where said mitochondrial dysfunction causes one or more of a neurodegenerative disease, a cardiovascular disease, a type of diabetes, a metabolic syndrome, an autoimmune disease, a neurobehavioral disease, a psychiatric disease, a gastrointestinal disorder, a fatiguing illness, a musculoskeletal disease, a cancer and a chronic infection.

7. The method of claim 6 where said neurodegenerative disease comprises Alzheimer's disease, Parkinson's disease, Huntington's disease, amyotrophic lateral sclerosis (ALS) and Friedreich's ataxia.

8. The method of claim 6 where said cardiovascular disease is a vascular condition comprising atherosclerosis.

9. The method of claim 6 where said autoimmune disease comprises multiple sclerosis, systemic lupus erythematosus and Type 1 diabetes.

10. The method of claim 6 where said neurobehavioral disease comprises an autism spectrum disorder, schizophrenia, a bipolar disorder, a mood disorder, depression, attention deficit hyperactivity disorder (ADHD) and post-traumatic stress disorder (PTSD).

11. The method of claim 6 where said fatiguing illness comprises chronic fatigue syndrome and a Gulf War illness.

12. The method of claim 6 where said musculoskeletal disease comprises fibromyalgia and skeletal muscle atrophy.

13. A diagnostic platform comprising:
(a) one or more reference models each able to predict a mitochondrial dysfunction in an unseen biomarker measurement made on a clinical sample obtained from an undiagnosed patient;
(b) one or more cell-cultures grown in vitro from one or more cell-lines and said mitochondrial dysfunction induced in said one or more cell-cultures by an introduction in one or more dosages of a mitochondrial inhibitor;
(c) said one or more reference models trained by a learning module based on reference biomarker measurements made from corresponding each of one or more reference samples drawn from said one or more cell-cultures at one or more times since said introduction, said learning module comprising a microprocessor executing program instructions stored in a non-transitory storage medium coupled to said microprocessor;
(d) one or more target subjects in whom said mitochondrial dysfunction is predicted by said one or more reference models based on one or more target biomarker measurements made on corresponding one or more target samples drawn in vivo from said one or more target subjects; and
(e) based on a statistically significant number of subjects from said one or more target subjects who are predicted to have said mitochondrial dysfunction in (d) above, an association developed by a targeting module between said mitochondrial dysfunction and a mitochondrial disease, said targeting module comprising a microprocessor executing program instructions stored in a non-transitory storage medium coupled to said microprocessor.

14. The platform of claim 13 further comprising a diagnosis module to predict by said one or more reference models said mitochondrial dysfunction in said undiagnosed patient based on said unseen biomarker measurement, and based on said association also said mitochondrial disease in said undiagnosed patient, said diagnosis module comprising a microprocessor executing program instructions stored in a non-transitory storage medium coupled to said microprocessor.

15. The platform of claim 14 further comprising a mass spectrometer to make one or more of said reference biomarker measurements, said target biomarker measurements and said unseen biomarker measurement.

16. The platform of claim 15 further comprising genomic data of said one or more target subjects obtained by one or more DNA sequencers and said mitochondrial disease is known to exist in said one or more target subjects based on said genomic data.

17. The platform of claim 13 wherein one or both of multiple linear regression and multiple logistic regression are used by said learning module.

18. The platform of claim 17 wherein a personalized targeted therapy for said undiagnosed patient is recommended based on said association of said mitochondrial dysfunction and said mitochondrial disease and on a known rescuer for said mitochondrial dysfunction.

19. The platform of claim 18 wherein said diagnosis module produces a diagnostic ranking for said undiagnosed patient, said diagnostic ranking containing a rank of said mitochondrial dysfunction, said mitochondrial disease, said mitochondrial inhibitor and a rescuer that is known to alleviate the effects of said mitochondrial dysfunction.

20. The platform of claim 18 wherein said mitochondrial dysfunction causes one or more of a neurodegenerative disease, a cardiovascular disease, a type of diabetes, a metabolic syndrome, an autoimmune disease, a neurobehavioral disease, a psychiatric disease, a gastrointestinal disorder, a fatiguing illness, a musculoskeletal disease, a cancer and a chronic infection.

21. The platform of claim 18 wherein said neurodegenerative disease comprises Alzheimer's disease, Parkinson's disease, Huntington's disease, amyotrophic lateral sclerosis (ALS) and Friedreich's ataxia.

22. The platform of claim 18 wherein said cardiovascular disease is a vascular condition comprising atherosclerosis.

23. The platform of claim 13 wherein said association exposes a correlation between said mitochondrial inhibitor and a genetic pattern of said undiagnosed patient.

24. A diagnostic system comprising:
(a) one or more reference models each able to predict a mitochondrial dysfunction in an unseen biomarker measurement made on a clinical sample obtained from an undiagnosed patient;
(b) one or more cell-cultures grown in vitro from one or more cell-lines and said mitochondrial dysfunction induced in said one or more cell-cultures by an introduction in one or more dosages of a mitochondrial inhibitor;
(c) said one or more reference models trained by a learning module based on reference biomarker measurements made from corresponding each of one or more reference samples drawn from said one or more cell-cultures at one or more times since said introduction, said learning module comprising a microprocessor executing program instructions stored in a non-transitory storage medium coupled to said microprocessor;
(d) one or more target subjects in whom said mitochondrial dysfunction is predicted by said one or more reference models based on one or more target biomarker measurements made on corresponding one or more target samples drawn in vivo from said one or more target subjects;
(e) based on a statistically significant number of subjects from said one or more target subjects who are predicted to have said mitochondrial dysfunction in (d) above, an association developed by a targeting module between said mitochondrial dysfunction and a mitochondrial disease, said targeting module comprising a microprocessor executing program instructions stored in a non-transitory storage medium coupled to said microprocessor;
(f) one or more mass spectrometers that are used to make at least one of said reference biomarker measurements, said target biomarker measurements and said unseen biomarker measurement; and
(g) one or more DNA sequencing devices used to obtain sequenced genomic data of said one or more target subjects, and said mitochondrial disease is known to exist in said one or more target subjects based on said genomic data.
